(19) United States
(12) Patent Application Publication
(10) Pub. No.: US 2011/0189208 A1
(43) Pub. Date: Aug. 4, 2011

(54) TB VACCINE

(76) Inventors: Andrew William Heath, Sheffield (GB); Evy De Leenheer, Sheffield (GB)

(21) Appl. No.: 13/120,414

(22) PCT Filed: Sep. 22, 2009
(86) PCT No.: PCT/GB09/02242

§ 371 Date: Mar. 22, 2011

(30) Foreign Application Priority Data
Sep. 24, 2008 (GB) 0817480.7
Jun. 11, 2009 (GB) 0910001.7

(51) Int. Cl. A61K 39/04; A61K 39/395; A61P 31/06; A61P 37/04

(52) U.S. Cl. 424/178.1; 424/190.1; 424/248.1

(57) ABSTRACT

The invention relates to a vaccine useful in therapy and prevention of mycobacterial infections.

The invention relates to a vaccine useful in therapy and prevention of mycobacterial infections, such as tuberculosis and related bacterial infections, in particular for the treatment of subjects that are immune suppressed.

Tuberculosis [TB] is caused mycobacteria, typically by Mycobacterium tuberculosis. Tuberculosis typically attacks the lungs and can also affect other essential functions, for example the central nervous system and skeleton and joints. It is common for subjects to act as carriers of disease and a large number of carriers are asymptomatic. However around 10% of these latent infections progress to full TB which if left untreated is fatal to a large number of diseased subjects. In particular, subjects that are immune suppressed because of, for example HIV infection, are particularly susceptible to TB. In addition there is the emergence of antibiotic resistant forms of Mycobacterium spp which is compounding the problems associated with TB. TB can be carried by non-human mammals and can cause serious disease in livestock. For example, M. bovis causes TB in cattle. The problems associated with controlling TB mean that there is a continual need to develop alternative means to control infection and treat those suffering from TB and related conditions or to protect subjects that are susceptible to TB.

Vaccines protect against a wide variety of infectious diseases. Many vaccines are produced by inactivated or attenuated pathogens which are injected into an individual. The immunised individual responds by producing both a humoral and cellular response. For example, some influenza vaccines are made by inactivating the virus by chemical treatment with formaldehyde, likewise the Salk polio vaccine comprises whole virus inactivated with propionolactone. For many pathogens chemical or heat inactivation, while it may give rise to vaccine immunogens that confer protective immunity, also gives rise to side effects such as fever and injection site reactions. In the case of bacteria, inactivated organisms tend to be so toxic that side effects have limited the application of such crude vaccine immunogens (e.g. the cellular pertussis vaccine). Many modern vaccines are therefore made from protective antigens of the pathogen, separated by purification or molecular cloning from the materials that give rise to side-effects. These latter vaccines are known as ‘subunit vaccines’.

The development of subunit vaccines has been the focus of considerable research in recent years. The emergence of new pathogens and the growth of antibiotic resistance have created a need to develop new vaccines and to identify further candidate molecules useful in the development of subunit vaccines. Likewise the discovery of novel vaccine antigens from genomic and proteomic studies is enabling the development of new subunit vaccine candidates, particularly against bacterial pathogens and cancers. However, although subunit vaccines tend to avoid the side effects of killed or attenuated pathogen vaccines, their ‘pure’ status means that subunit vaccines do not always have adequate immunogenicity. Many candidate subunit vaccines have failed in clinical trials in recent years that might otherwise have succeeded were a suitable adjuvant available to enhance the immune response to the purified antigen. An adjuvant is a substance or procedure which augments specific immune responses to antigens by modulating the activity of immune cells.

The receptor CD40 plays an important co-stimulatory role in the activation of B-cells during the cognate interaction of antigen-specific T and B-cells that gives rise to an antibody response. The CD40 signal is pivotal to the expression of T cell help and immunoglobulin class-switching in both humans and mice. In addition to its importance in T and B-cell interactions, ligation of CD40 is also very important in activation of macrophages and of dendritic cells to express co-stimulatory antigens and thus in the generation of helper T cell priming by these antigen-presenting cells. In recent studies we have shown that ligation of CD40 by antibodies can effectively replace the CD40 signals ordinarily made during intercellular interaction in the immune response (see WO03/063899; WO2004/052396 and WO2004/041866. Vaccines increasingly are required to be ‘multivalent’ (e.g. containing antigens from several different strains of a pathogen or containing multiple proteins from a single pathogen that are additive or synergistic in the protective immune response they generate (as is the case for a number of vaccines under development—e.g. for H. pylori, tuberculosis etc.).

We herein disclose a multivalent vaccine useful in the treatment and prevention of diseases caused by mycobacterial infections and in particular in subjects that are immune suppressed and susceptible to bacterial infection, in particular mycobacterial infections.

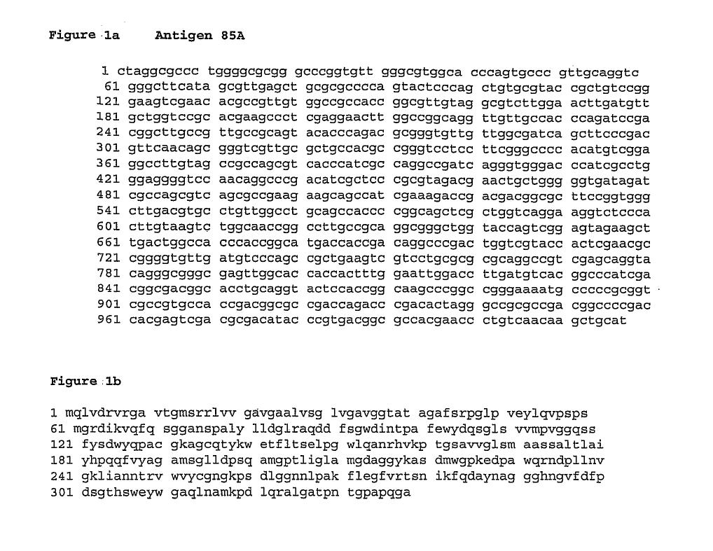

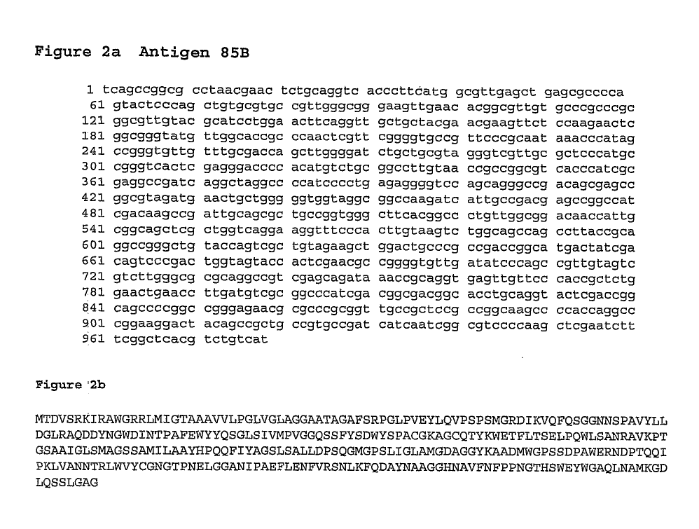

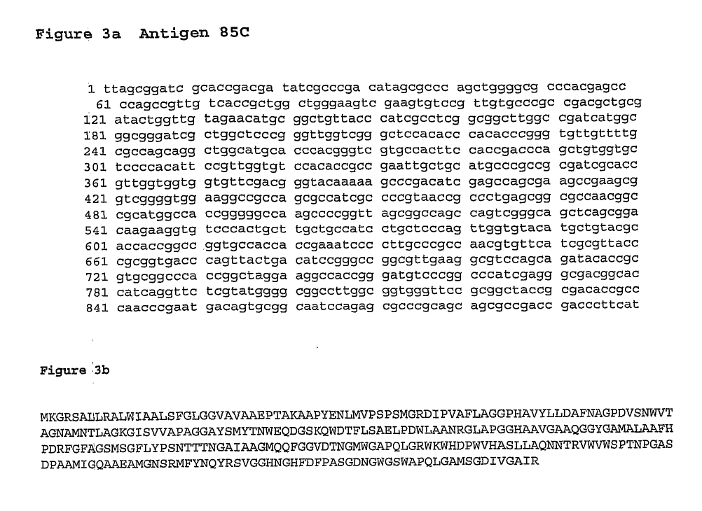

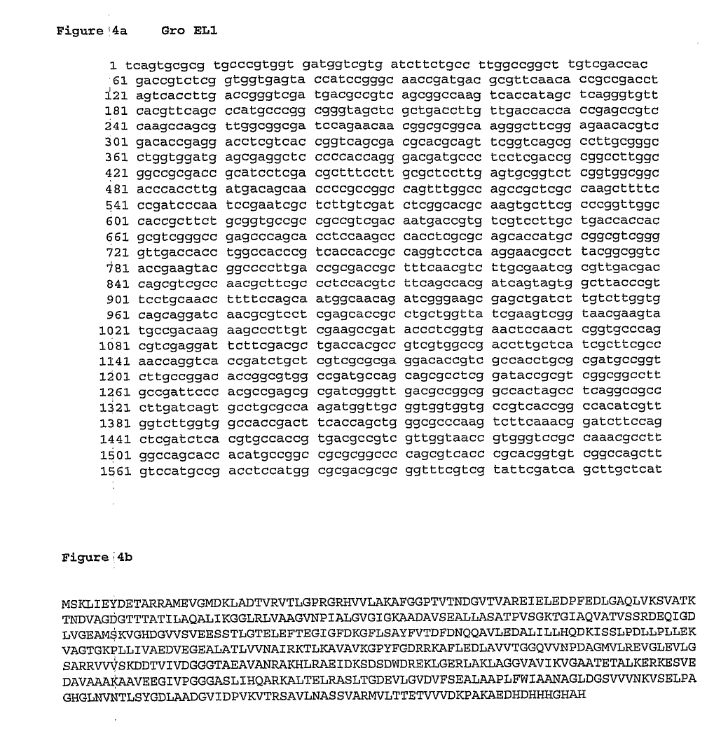

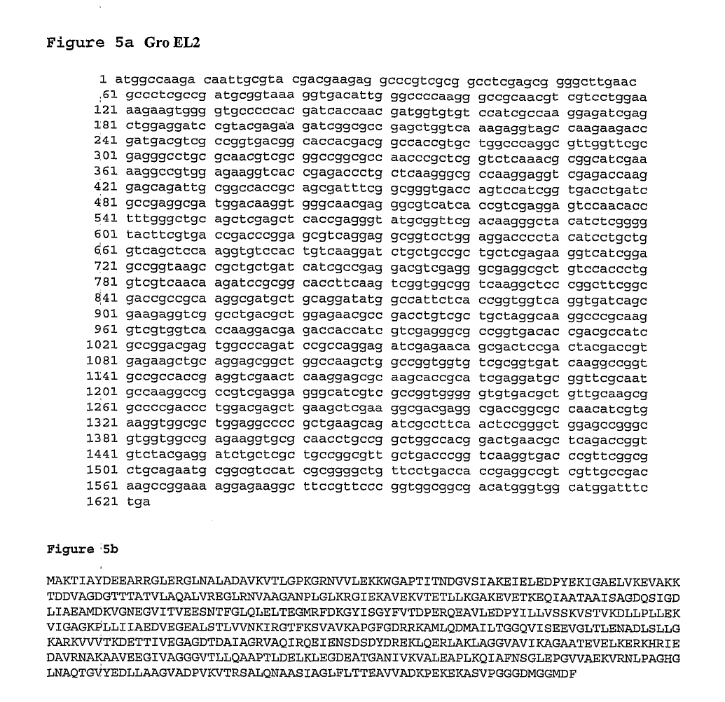

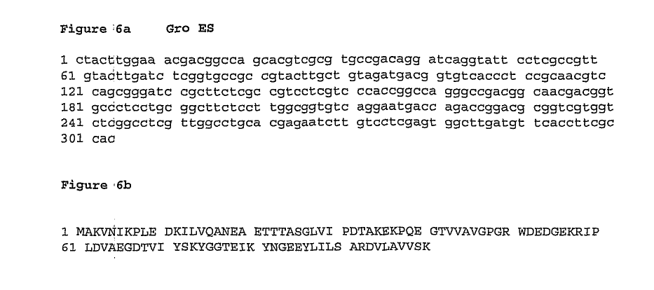

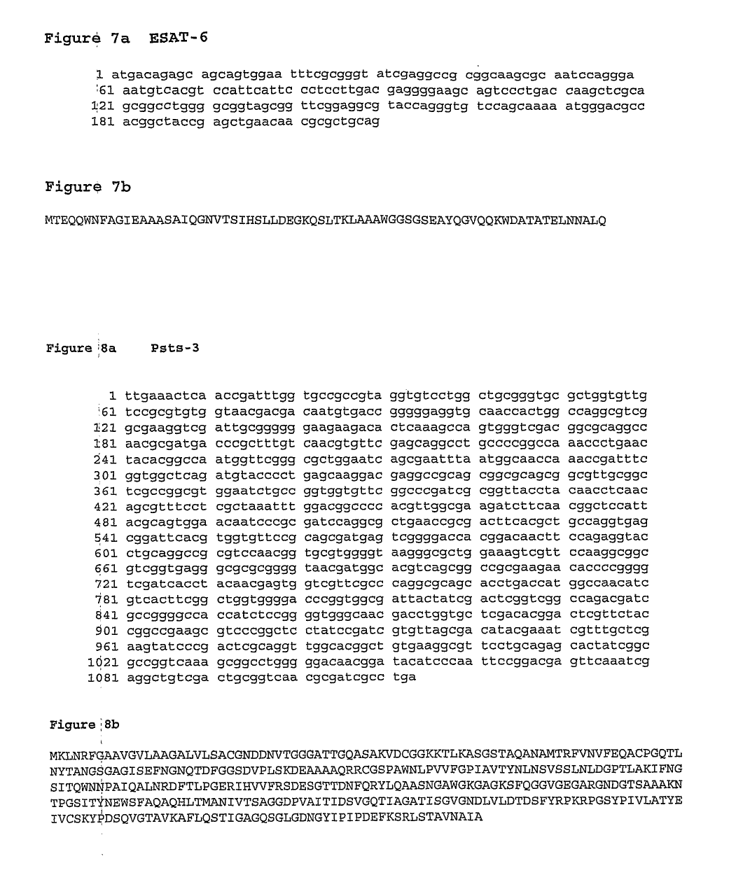

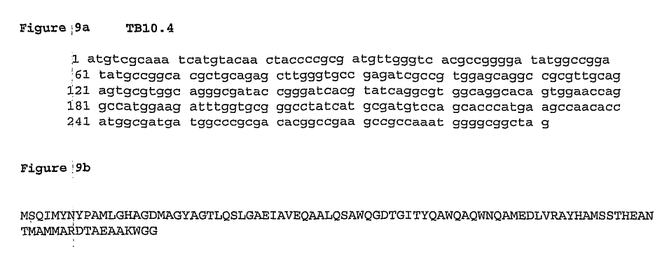

According to an aspect of the invention there is provided a vaccine composition comprising a nucleic acid or polypeptide selected from the group consisting of:
i) a nucleic acid molecule as represented by the nucleic acid sequence in FIG. 1a and/or FIG. 2a and/or FIG. 3a and/or FIG. 4a and/or FIG. 5a and/or FIG. 6a and/or FIG. 7a and/or FIG. 8a and/or FIG. 9a;
ii) a nucleic acid molecule that hybridizes to the nucleic acid molecule in (i) under stringent hybridization conditions wherein said nucleic acid encodes a polypeptide that has the activity associated with the mycobacterial proteins Ag85A, Ag85B, AgAg85C, GroEL, GroEL2, GroES ESAT-6, Psts-3 and TB 10.4;
iii) a polypeptide comprising an amino acid sequence as represented in FIGS. 1b and/or FIG. 2b and/or FIG. 3b and/or FIG. 4b and/or FIG. 5b and/or FIG. 6b and/or FIG. 7b and/or FIG. 8b and/or FIG. 9b or antigenic part thereof; and wherein said composition further comprises a nucleic acid molecule that encodes a CD40 ligand, or a polypeptide with CD40 ligand binding activity that binds and activates CD40 receptor expressed by antibody producing B-lymphocytes.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a preferred embodiment of the invention said vaccine comprises a polypeptide comprising an amino acid sequence as represented in FIG. 1b and/or FIG. 2b and/or FIG. 3b and/or FIG. 4b and/or FIG. 5b and/or FIG. 6b FIG. 7a and/or FIG. 8a and/or FIG. 9a.

In a preferred embodiment of the invention said polypeptide is associated with said CD40 ligand.

In a preferred embodiment of the invention said polypeptide is cross-linked to said CD40 ligand.

In a preferred embodiment of the invention said ligand is a CD40 monoclonal antibody or CD40 active fragment thereof.

Alternatively, said antibody is a chimeric antibody produced by recombinant methods to contain the variable region of said antibody with an invariant or constant region of a human antibody.

In a further alternative embodiment of the invention, said antibody is humanised by recombinant methods to combine the complimentarity determining regions of said antibody with both the constant (C) regions and the framework regions from the variable (V) regions of a human antibody.

Preferably said humanised monoclonal antibody to said polypeptide is produced as a fusion polypeptide in an expression vector suitably adapted for transfection or transformation of prokaryotic or eukaryotic cells.

In a preferred embodiment of the invention said ligand is an antibody fragment.

Various fragments of antibodies are known in the art, e.g. Fab, Fab₂, F(ab′)₂, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab₂ fragment comprises two joined Fab fragments. When these two fragments are joined by the immunoglobulin hinge region, a F(ab′)₂ fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region (domain) fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology (cited above). Molecular biology now allows direct synthesis (via expression in cells or chemically) of these fragments, as well as synthesis of combinations thereof. A fragment of an antibody or immunoglobulin can also have bispecific function as described above.

In a preferred embodiment of the invention there is provided a therapeutic vaccine composition according to the invention comprising at least one polypeptide, or an antigenic part thereof that is encoded by the Dos R regulon. Preferably said polypeptide is selected from the group consisting of: Rv2629, Rv80, Rv8, Rv570, Rv571c, Rv573c, Rv574c, Rv1734c, Rv1735c, Rv1736c, Rv1737c, Rv1812c, Rv1997, Rv1998c, Rv2003c, Rv2004c, Rv2005c, Rv2006, Rv2028c, Rv2625c, Rv2630, Rv2631, Rv3128c, Rv0079, Rv569, Rv572, Rv1738, Rv1813, Rv1996, Rv2007c, Rv2029c, Rv2030c, Rv2031c, Rv2032, Rv2623, Rv2624c, Rv2626, Rv2627c, Rv2628, Rv3126c, Rv3127, Rv3129, Rv3130, Rv3131, Rv3132, Rv3133c or Rv3134c.

In a preferred embodiment of the invention said polypeptide is represented by the amino acid sequence as represented in FIG. 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 or 54 or antigenic part thereof.

Preferably said composition further comprises Bacille Calmette Guerin [BCG].

In a preferred embodiment of the invention said CD40 monoclonal antibody is an isotype selected from the group consisting of: IgA, IgM, IgD, IgE and IgG.

Preferably said isotype is selected from the group consisting of: IgG1, IgG2, IgG3 and IgG4. More preferably said isotype is human IgG2 or IgG4.

In a preferred embodiment of the invention said antibody is a modified antibody wherein said modification reduces or abrogates the binding of said antibody to the B-cell receptor FcgammaR Ilb.

Preferably said modified antibody is an IgG antibody modified at C-g-2 domain of the heavy chain at asparagine 297 by deletion or substitution of said asparagine residue.

Alternatively said modified antibody is an IgG antibody modified at C-g-2 domain of the heavy chain at asparagine 265 by deletion or substitution of said asparagine residue.

Alternatively still said modified antibody is an antibody modified at C-g-2 domain of the heavy chain at proline 331 by substitution with a serine residue.

According to an aspect of the invention there is provided a vaccine composition comprising a Mycobacterium tuberculosis antigenic polypeptide crosslinked to a CD40 monoclonal antibody or CD40 active binding fragment thereof.

In a preferred embodiment of the invention said monoclonal antibody is an isotype selected from the group consisting of: IgA, IgM, IgD, IgE and IgG.

Preferably said isotype is selected from the group consisting of: IgG1, IgG2, IgG3 and IgG4. More preferably said isotype is human IgG2 or IgG4.

In a preferred embodiment of the invention said antibody is a modified antibody wherein said modification reduces or abrogates the binding of said antibody to the B-cell receptor Fc gamma R Ilb.

In a preferred embodiment of the invention said modified antibody is an IgG antibody modified at C-g-2 domain of the heavy chain at asparagine 297 by deletion or substitution of said asparagine residue.

Alternatively said modified antibody is an IgG antibody modified at C-g-2 domain of the heavy chain at asparagine 265 by deletion or substitution of said asparagine residue.

Alternatively still said modified antibody is an Ig antibody modified at C-g-2 domain of the heavy chain at proline 331 by substitution with a serine residue.

In a preferred embodiment of the invention said composition includes a second agent wherein said second agent is a second adjuvant or carrier.

The terms adjuvant and carrier are construed in the following manner. Some polypeptide or peptide antigens contain B-cell epitopes but no T cell epitopes. Immune responses can be greatly enhanced by the inclusion of a T cell epitope in the polypeptide/peptide or by the conjugation of the polypeptide/peptide to an immunogenic carrier protein such as key hole limpet haemocyanin or tetanus toxoid which contain multiple T cell epitopes. The conjugate is taken up by antigen presenting cells, processed and presented by human leukocyte antigens (HLAs/MHCs) class II molecules. This allows T cell help to be given by T cell's specific for carrier derived epitopes to the B cell which is specific for the original antigenic polypeptide/peptide. This can lead to increase in antibody production, secretion and isotype switching.

An adjuvant is a substance or procedure which augments specific immune responses to antigens by modulating the activity of immune cells. Examples of adjuvants include, by example only, agonsitic antibodies to co-stimulatory molecules, Freunds adjuvant, muramyl dipeptides, and liposomes. An adjuvant is therefore an immunomodulator. A carrier is an immunogenic molecule which, when bound to a second molecule augments immune responses to the latter.

According to a further aspect of the invention there is provided a vector comprising a nucleic acid sequence selected from the group consisting of:
i) a nucleic acid molecule as represented by the nucleic acid sequence in FIG. 1a and/or FIG. 2a and/or FIG. 3a and/or FIG. 4a and/or FIG. 5a and/or FIG. 6a and/or FIG. 7a and/or FIG. 8a and/or FIG. 9a;
i) a nucleic acid molecule that hybridizes to the nucleic acid molecule in (i) under stringent hybridization conditions wherein said nucleic acid encodes a polypeptide that has the activity associated with the mycobacterial proteins Ag85A, Ag85B, AgAg85C, GroEL, GroEL 2, GroES, ESAT-6, Psts-3 and TB 10.4; wherein said vector further includes a nucleotide sequence that encodes a CD40 ligand that binds and activates CD40 receptor expressed by antibody producing B-lymphocytes.

According to a further aspect of the invention there is provided a cell transfected or transformed with the vector according to the invention.

According to a further aspect of the invention there is provided a method to treat a subject that is infected with or has a predisposition to a mycobacterial infection comprising administering to said subject an effective amount of a vaccine composition wherein said composition comprises a nucleic acid molecule or polypeptide selected from the group consisting of:
ii) a nucleic acid molecule as represented by the nucleic acid sequence in FIG. 1a and/or FIG. 2a and/or FIG. 3a and/or FIG. 4a and/or FIG. 5a and/or FIG. 6a and/or FIG. 7a and/or FIG. 8a and/or FIG. 9a;
iii) a nucleic acid molecule that hybridizes to the nucleic acid molecule in (i) under stringent hybridization conditions wherein said nucleic acid encodes a polypeptide that has the activity associated with the mycobacterial proteins Ag85A, 85B, Ag85C, GroEL, GroEL 2, GroES, ESAT-6, Psts-3 and TB 10.4;
iv) a polypeptide comprising an amino acid sequence as represented in FIGS. 1b and/or FIG. 2b and/or FIG. 3b and/or FIG. 4b and/or FIG. 5b and/or FIG. 6b and/or FIG. 7b and/or FIG. 8b and/or FIG. 9b or antigenic part thereof; and wherein said composition further comprises a nucleic acid molecule that encodes a CD40 ligand, or a polypeptide with CD40 ligand binding activity that binds and activates CD40 receptor expressed by antibody producing B-lymphocytes.

In a preferred method of the invention said mycobacterial infection is tuberculosis.

In a preferred method of the invention said mycobacterial infection is leprosy.

In a preferred method of the invention said subject is immune compromised.

In a further preferred method of the invention said subject is infected with human immune deficiency virus [HIV].

In an alternative preferred method of the invention said subject is a livestock animal, for example bovine species.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1a is the nucleic acid sequence of Ag85A; FIG. 1b is the amino acid sequence of Ag85A;

FIG. 2a is the nucleic acid sequence of Ag85A; FIG. 2b is the amino acid sequence of Ag85B;

FIG. 3a is the nucleic acid sequence of AgAg85C; FIG. 3b is the amino acid sequence of Ag85C;

FIG. 4a is the nucleic acid sequence of GroEL; FIG. 4b is the amino acid sequence of GroEL;

FIG. 5a is the nucleic acid sequence of GroEL 2; FIG. 5b is the amino acid sequence of GroEL 2;

FIG. 6a is the nucleic acid sequence of GroES; FIG. 6b is the amino acid sequence of GroES;

FIG. 7a is the nucleic acid sequence of ESAT-6; FIG. 7b is the amino acid sequence of ESAT-6;

FIG. 8a is the nucleotide sequence of Psts-3; FIG. 8b is the amino acid sequence of Psts 3;

FIG. 9a is the nucleotide sequence of TB 10.4; FIG. 9b is the amino acid sequence of TB10.4;

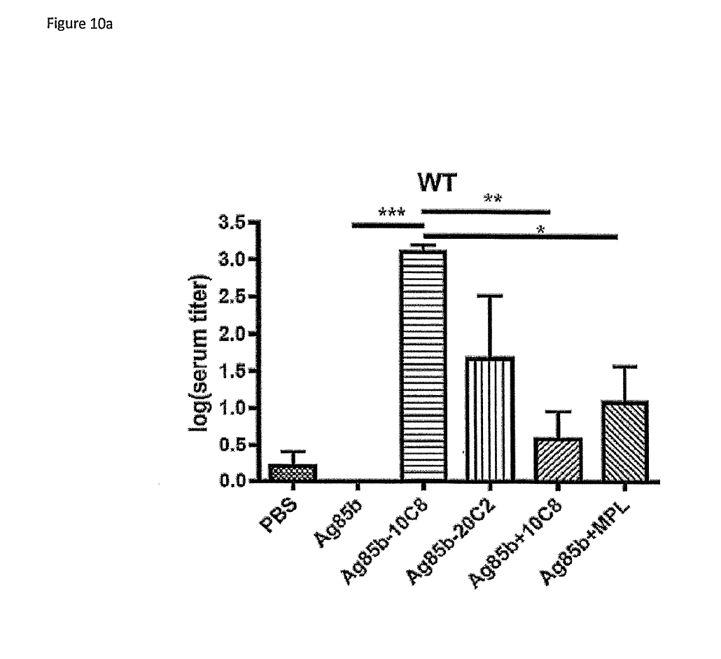

FIG. 10a shows enhanced antibody response against Ag85B induced by conjugation to anti-CD40 antibody; FIG. 10b is an identical experiment to FIG. 10a except for the depletion of CD4 cells;

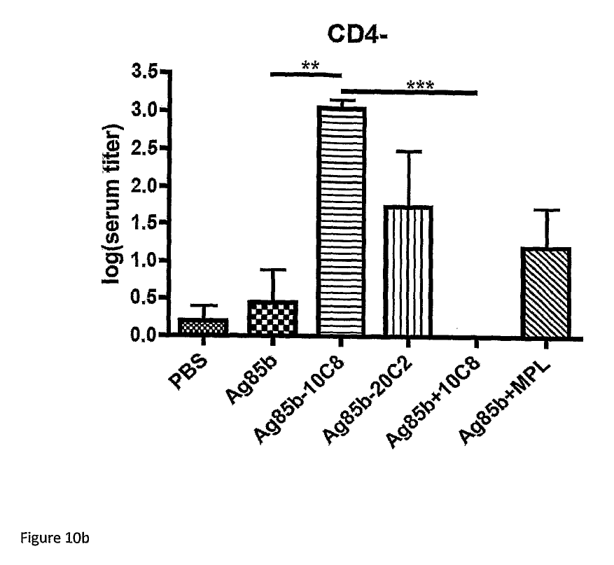

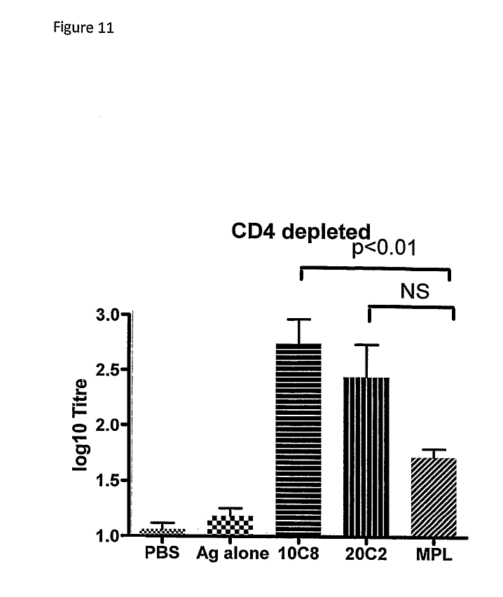

FIG. 11 as described in FIG. 10b but using AgAg85A as the vaccine antigen;

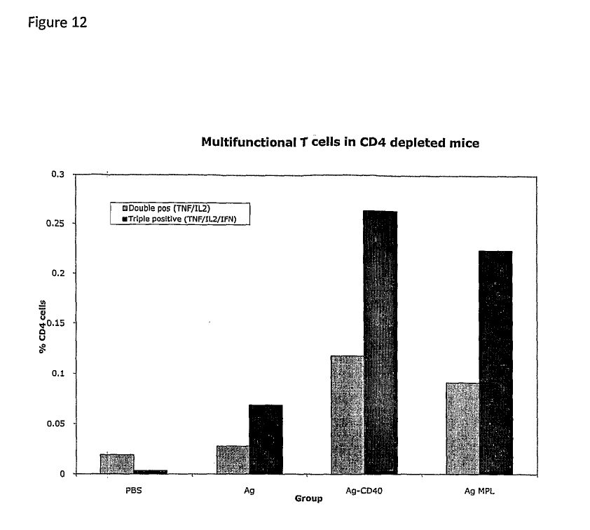

FIG. 12 describes multifunctional T cells in CD40-TB immunised mice;

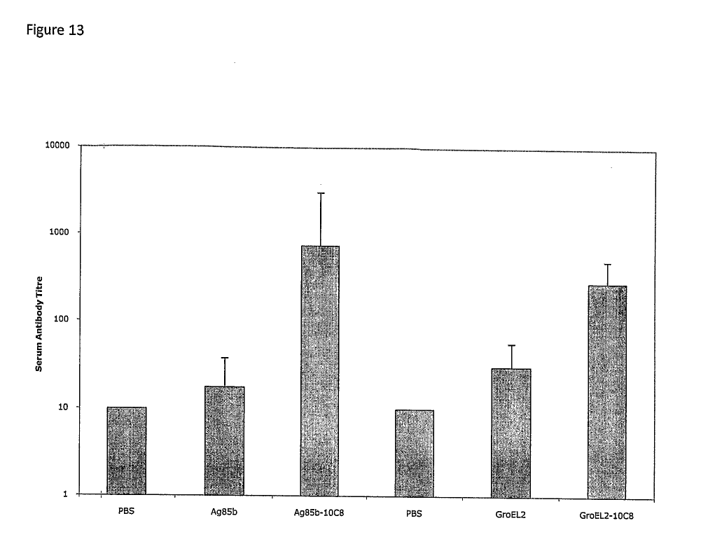

FIG. 13 shows enhanced antibody responses against Ag85B and GroEL2 by conjugation to CD40 mAb;

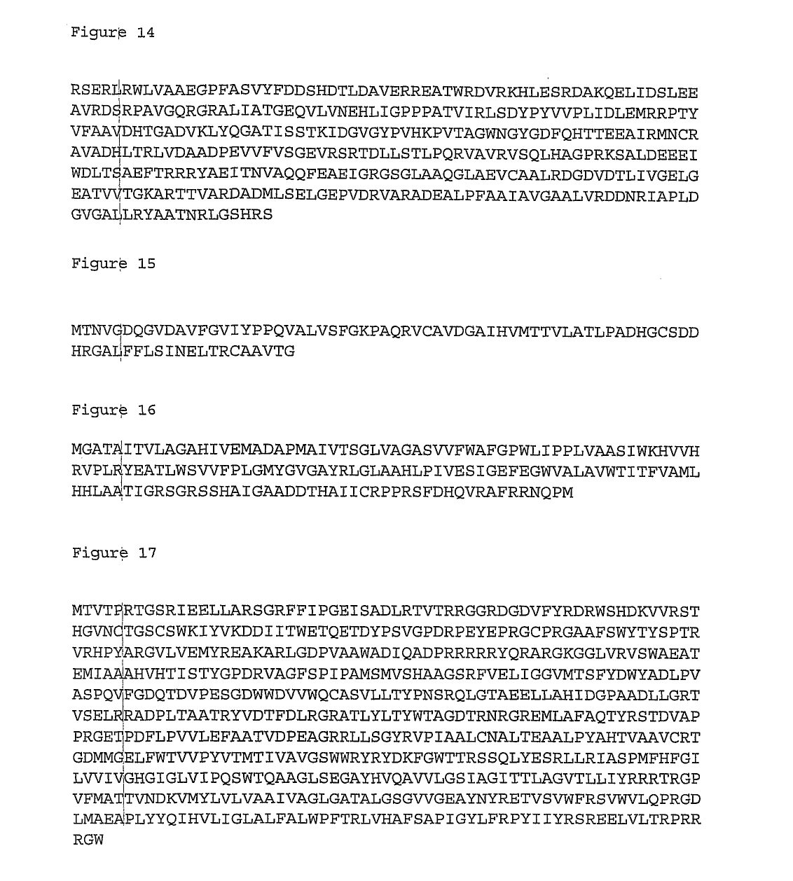

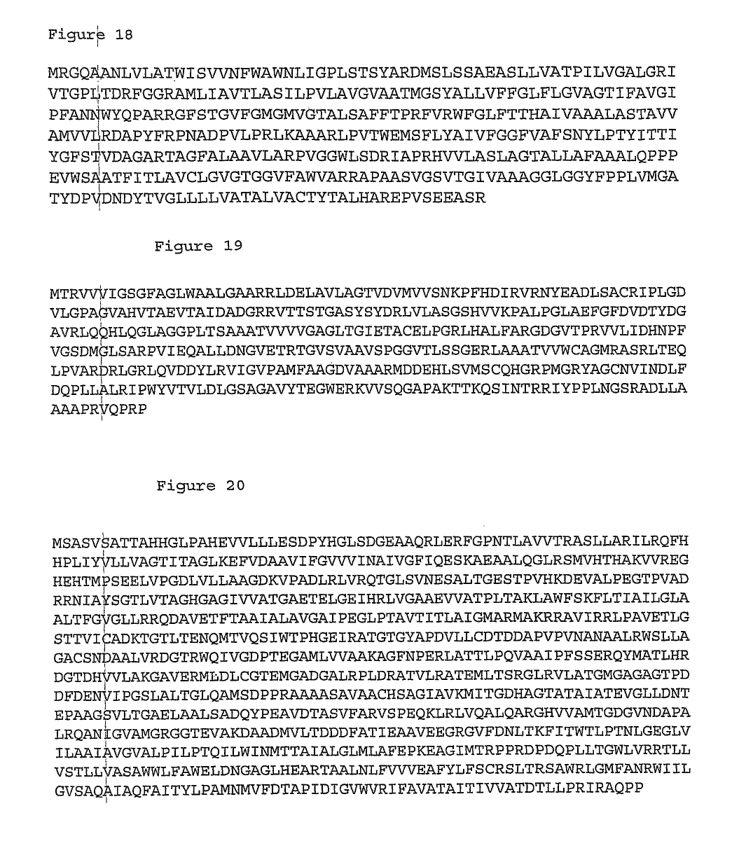

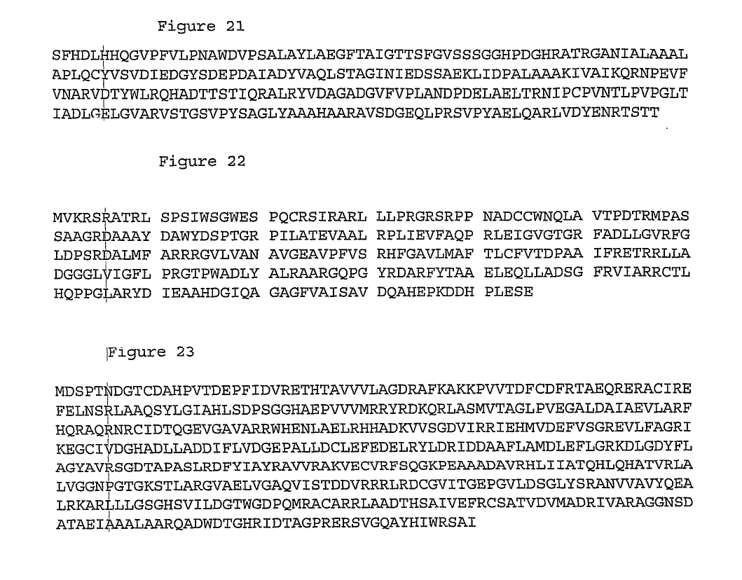

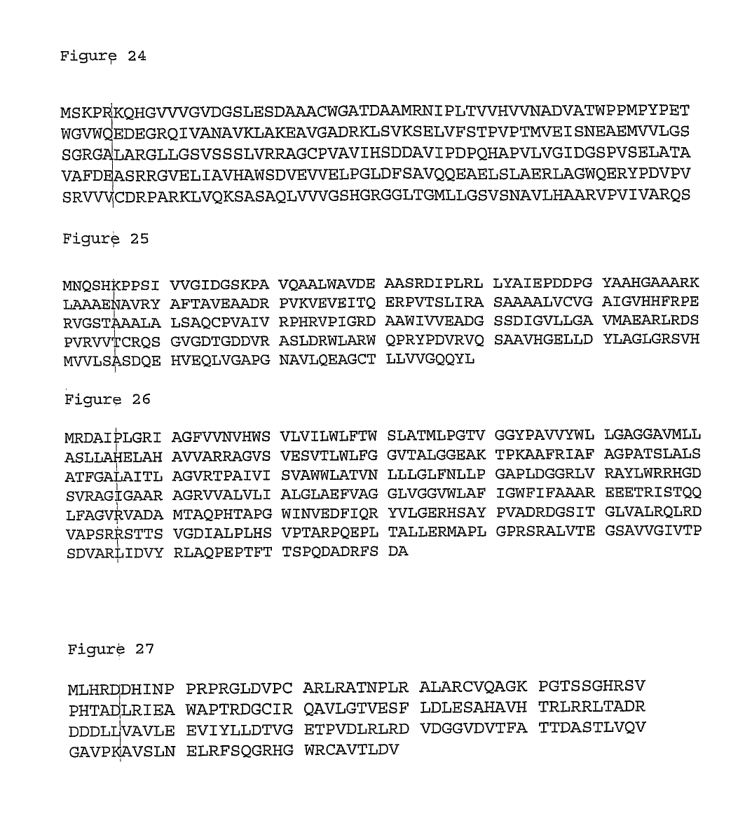

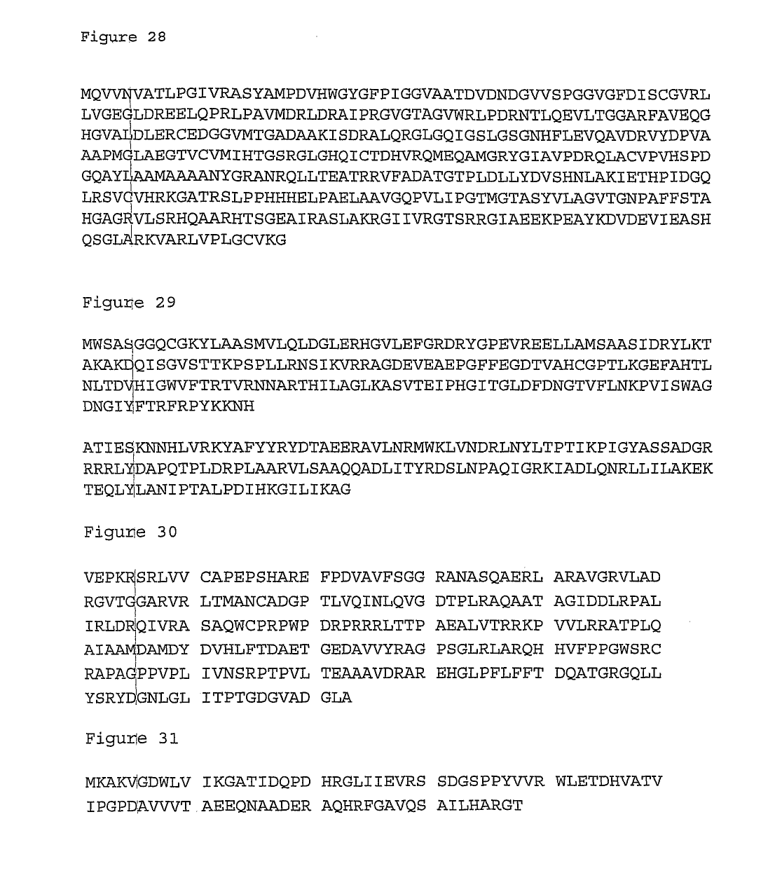

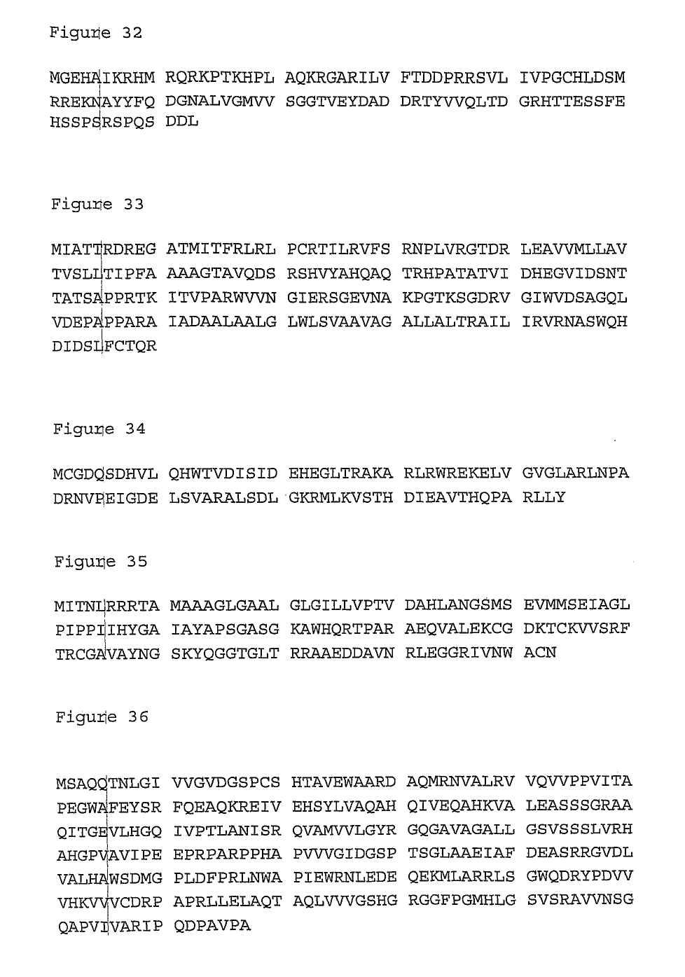

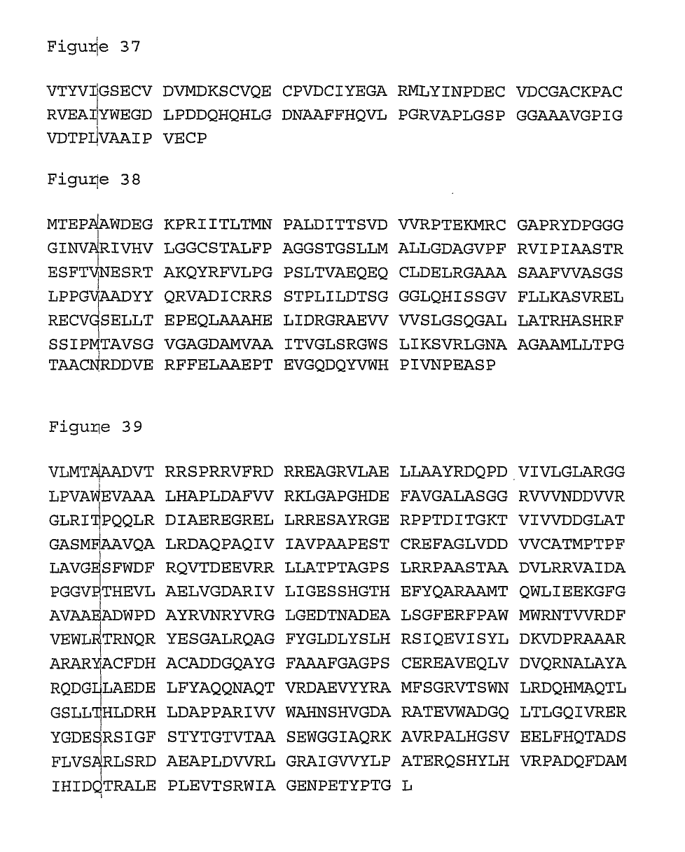

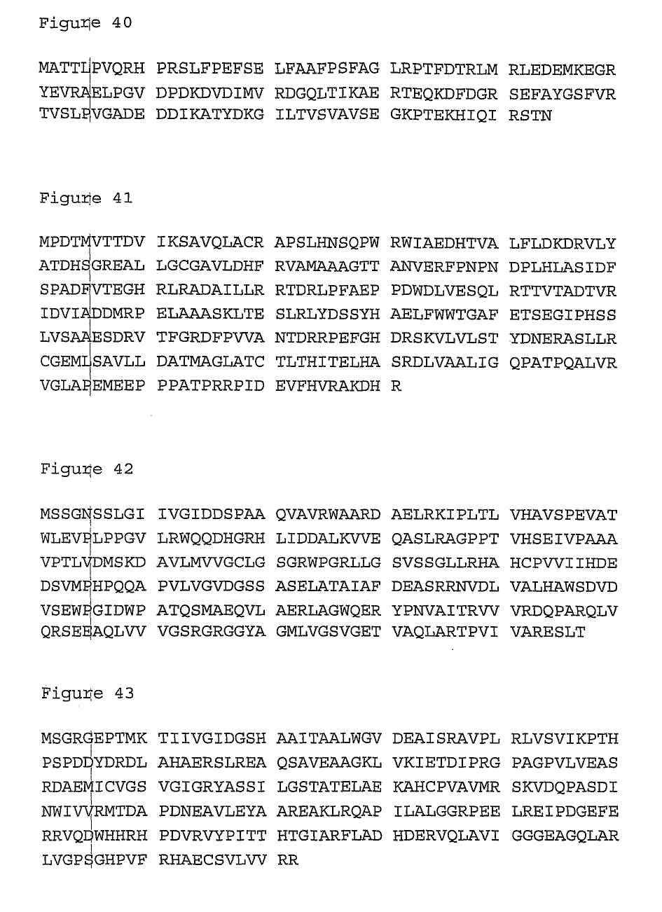

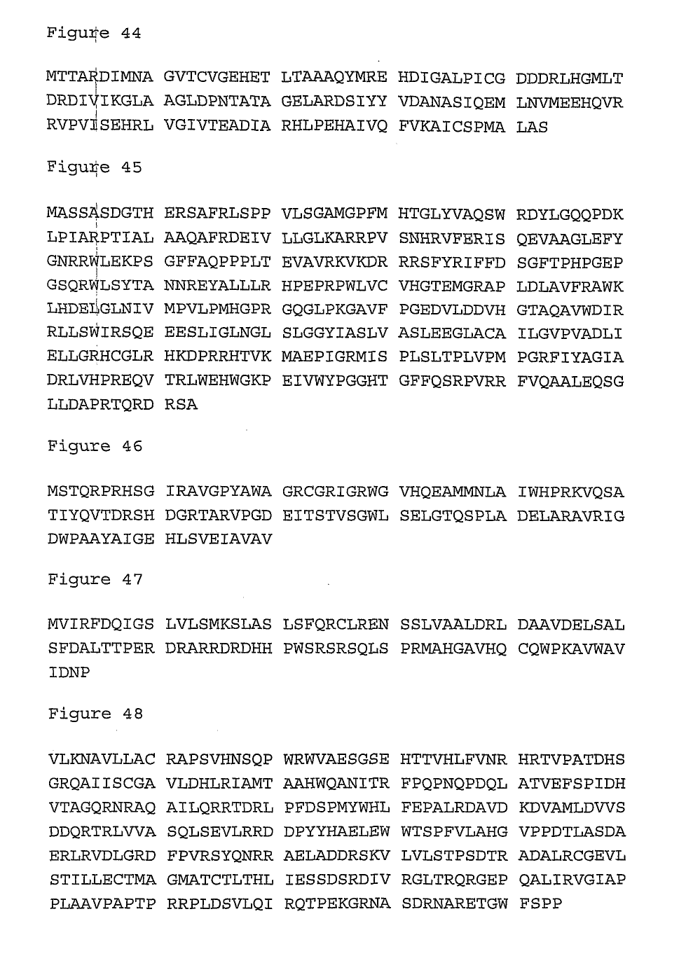

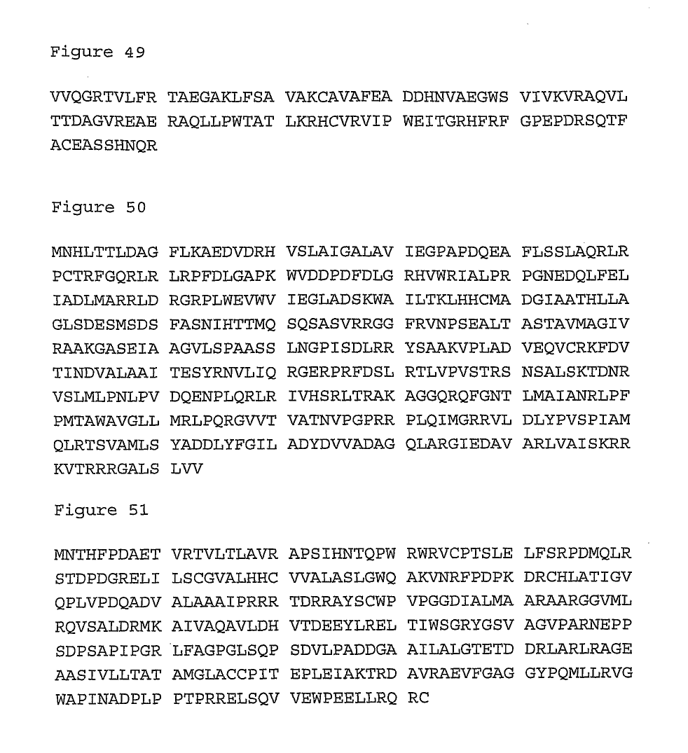

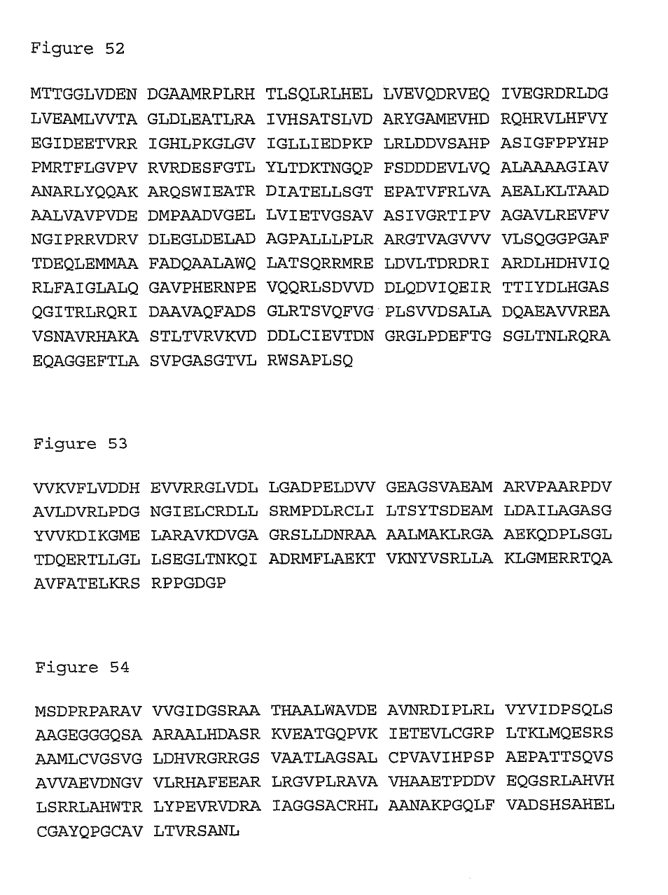

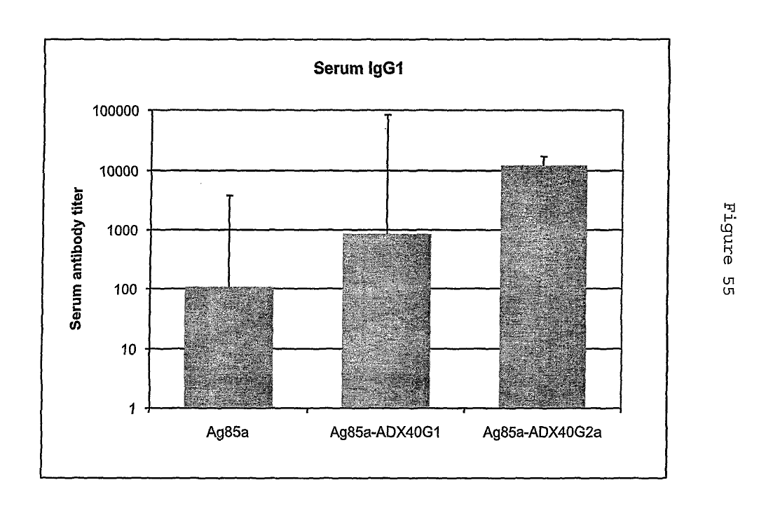

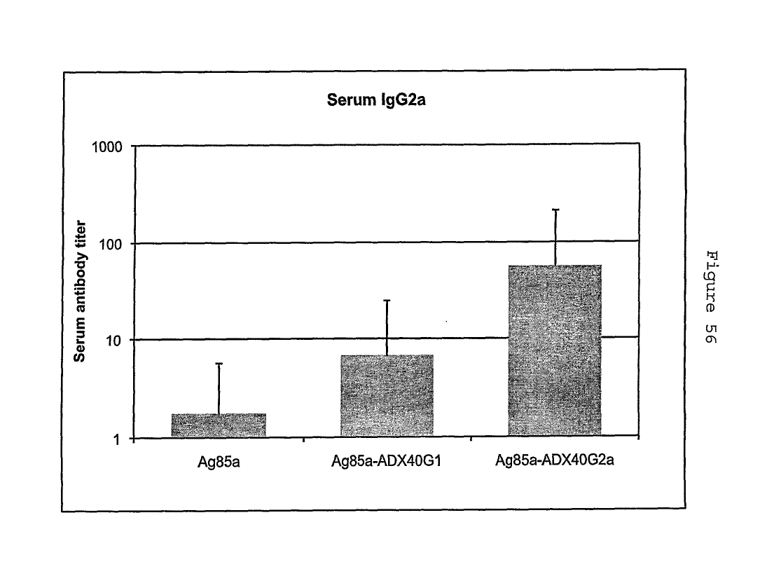

FIG. 14-54 represents the amino acid sequences of selected DosR encoded polypeptides

FIG. 55 illustrates serum IgG1 levels measured after immunization of rats with CD40/Ag85A conjugates; and

FIG. 56 illustrates serum IgG2a levels measured after immunization of rats with CD40/Ag85A conjugates.

### MATERIALS AND METHODS

### TB Antigens

The Mycobacterium tuberculosis antigens are produced as recombinant proteins expressed by E. coli. In the examples we have shown the proteins have H is (6) tags, and have been purified on Nickel columns

### Conjugation of Anti-CD40 to TB Antigens

The technique is an adaptation of that described in Barr et al {Barr et al., 2003, Immunology, 109, 87-92}. Of course there are many different cross-linking methods and reagents available. Many of these are described in the catalogue of InVitrogen (Molecular Probes), and many of these techniques would be suitable.

### Materials

Use fresh Sulfo-SMCC to derivatise TB antigens
Use fresh SATA to activate antibodies
Hydroxylamine buffer (50 ml): 0.5 M Hydroxylamine
25 mM EDTA in PBS
pH 7.2-7.5.
Dissolve 1.74 g hydroxylamine.HCl and EDTA (0.475 g of tetrasodium salt or 0.365 g of disodium salt) in 40 ml PBS. Add ultrapure water to a final volume of 50 ml and adjust pH to 7.2-7.5 with NaOH.
   Amicon Ultra-4 filters:
add 3 ml PBS to 30 kD centrifuge filter
spin at 400 g for 15 min, RT
remove any remaining PBS from insert

### Buffer Exchange TB Antigens

Buffer exchange TB antigens into PBS either by dialysis or using an Amicon Ultra-4 spinfilter—12 ml total (3 spins with 4 ml PBS) is usually sufficient; make sure not to over-concentrate the protein to avoid aggregation

Resuspend final retentate at 1-4 mg/ml in PBS

### Block —SH Groups in TB Antigen

dissolve N-ethyl-maleimide at 25 mg/ml in dH2O
add equal amount (mg) of NEM to TB antigen
incubate for 2 hours at RT on shaker/rotator
   Activation of TB Antigens with Sulfo-SMCC
dissolve 2 mg Sulfo-SMCC in 600 μl milliQ; if this does not dissolve easily, warm to 50° C.
add 60 μl Sulfo-SMCC solution to 1 ml TB antigen solution (1-4 mg/ml)
incubate for 1 hour at RT on shaker/rotator
transfer solution to an Amicon Ultra-4 spinfilter
add up to 4 ml PBS, spin at 400 g for 10-20 min or until retentate ˜0.5 ml (avoid over-concentrating the protein solution!); repeat 3 times
resuspend retentate in 0.3-0.5 ml PBS

### Activation of Anti-CD40 Antibody

dissolve 6-8 mg SATA per 500 μl DMSO
add 10 μl SATA-solution to 1 ml anti-CD40 (2-10 mg/ml)
incubate for 30 minutes at RT
transfer solution to an Amicon Ultra-4 spinfilter
add up to 4 ml PBS, spin at 400 g for 10-20 min or until retentate ˜0.5 ml (avoid over-concentrating the protein solution!); repeat 3 times
   (At this stage the SATA-treated protein can be stored indefinitely at −20 C for later use)

### Continued Protocol:

resuspend 1 mg antibody at 1 mg/ml
add 100 μl hydroxylamine buffer to 1 mg antibody
incubate for 2 hours at RT on mixer/rotator

### Conjugation

mix 1 mg maleimide-activated TB antigen (0.3-0.5 ml) with 1 mg sulfhydryl-anti-CD40 (1.1 ml)
incubate overnight at 4° C. on shaker/rotator
make up a fresh stock solution of 500 mM L-cysteine solution in MilliQ
add L-cysteine to the conjugated proteins at a final concentration of 50 mM in order to stop the reaction
incubate for 15 min at RT
transfer solution to an Amicon Ultra-4 spinfilter
add up to 4 ml PBS, spin at 400 g for 10-20 min or until retentate ˜0.5 ml (avoid over-concentrating the protein solution!); repeat 3 times
resuspend conjugate in 1 ml PBS
store conjugate at 4 C; if stored for a prolonged length of time, add 0.01% sodium azide

### Elisa Assay for Antibody Against TB Antigens

The ELISA assay was performed as described previously for HSV antigen and ovalbumin {Barr et al., 2003, Immunology, 109, 87-92} with various TB antigens used to coat the plates rather than HSV gD or Ovalbumin.

### Examples

C57/bl6 Mice were immunised once with 10 μg of CD40 mab (1008) or isotype control (20C2) conjugated to Ag85B, or with 10 μg Ag85B plus 10 μg 1008, or Monophosphoryl lipid A (Sigma) or Ag85B alone. After 12 days mice were bled and sera assayed by ELISA for antibody against Ag85B. Conjugation to the CD40 mAb induced a much stronger antibody response against Ag85B following a single immunisation. *** p<0.001, **p<0.005, * p<0.05.

FIG. 10b as above, except C57bl/6 mice were depleted of CD4 cells by i.p injection of the anti-CD4 antibody YTS191.1 as described previously {Dullforce et al., 1998, Nat Med, 4, 88-91}. CD4 cell counts in the blood were monitored, and the mice were immunised at the point where counts were lower than the threshold defining AIDS in humans.

Mice were depleted of CD4 cells and immunised with Ag85A conjugates as described in FIG. 11. Mice were boosted with 10 μg Ag85A alone after 13 days, and 12 days after the boost spleens were removed, red cells depleted and splenocytes incubated with Ag85A (10 μg/ml) in medium for x h, followed by intracellular cytokine staining (as described by Darrah et al {Darrah et al., 2007, Nat Med, 13, 843-50}, see FIG. 12.

In a separate experiment to that shown in FIG. 10, mice were immunised with 10 μg Ag85B or GroEL2 conjugated to CD40 mAb (1008), or antigen alone, and bled at 15 days post-immunisation. Antibody titres against Ag85B and GroEL2 were assessed by ELISA; see FIG. 13.

FIG. 55 illustrates antibody responses (mouse IgG1) against Ag85A induced by a single immunisation with rat IgG2a or IgG1-CD40 mAb conjugates. Mice were immunised with 10 μg of Ag85A-CD40 mAb conjugate (Ag85a-ADX40G2a and Ag85aADX40G1) or 5 μg of Ag85A alone (Ag85a) and serum antibody titres assessed by ELISA at day 14.

FIG. 56 illustrates antibody responses (mouse IgG2a) against Ag85A induced by a single immunisation with rat IgG2a or IgG1-CD40 mAb conjugates. Mice were immunised with 10 μg of Ag85A-CD40 mAb conjugate (Ag85a-ADX40G2a and Ag85aADX40G1) or 5 μg of Ag85A alone (Ag85a) and serum antibody titres assessed by ELISA at day 14.

1. A prophylatic or therapeutic vaccine composition comprising a polypeptide selected from the group consisting of:
a polypeptide comprising an amino acid sequence as represented in FIG. 1b and/or FIG. 2b and/or FIG. 3b and/or FIG. 4b and/or FIG. 5b and/or FIG. 6b and/or FIG. 7b and/or FIG. 8b and/or FIG. 9b or antigenic part thereof; and wherein said composition further comprises a polypeptide with CD40 ligand binding activity that binds and activates CD40 receptor expressed by antibody producing B-lymphocytes and wherein said polypeptide is cross-linked to said CD40 ligand.

2.-4. (canceled)

5. The composition according to claim 1, wherein said ligand is a CD40 monoclonal antibody or CD40 active fragment thereof.

6. The composition according to claim 5, wherein said antibody is a chimeric antibody.

7. The composition according to claim 5, wherein said antibody is a humanised antibody.

8. The composition according to claim 5 wherein said ligand is an antibody fragment.

9. The composition of claim 1, wherein said composition includes a second agent wherein said second agent is an adjuvant or carrier.

10. The composition according to claim 9, wherein said second agent is a TLR agonist.

11. The composition according to claim 10, wherein said TLR agonist is polyinosinic-polycytidylic acid (poly I; C), monophosphoryl lipid A, CpG containing double stranded DNA, or flagellin.

12. The composition of claim 1, comprising at least one polypeptide, or an antigenic part thereof that is encoded by the Dos R regulon.

13. A therapeutic vaccine comprising the composition according to claim 12 wherein said polypeptide is selected from the group consisting of: Rv2629, Rv80, Rv8, Rv570, Rv571c, Rv573c, Rv574c, Rv1734c, Rv1735c, Rv1736c, Rv1737c, Rv1812c, Rv1997, Rv1998c, Rv2003c, Rv2004c, Rv2005c, Rv2006, Rv2028c, Rv2625c, Rv2630, Rv2631, Rv3128c, Rv0079, Rv569, Rv572, Rv1738, Rv1813, Rv1996, Rv2007c, Rv2029c, Rv2030c, Rv2031c, Rv2032, Rv2623, Rv2624c, Rv2626, Rv2627c, Rv2628, Rv3126c, Rv3127, Rv3129, Rv3130, Rv3131, Rv3132, Rv3133c or Rv3134c.

14. The therapeutic vaccine according to claim 13, wherein said polypeptide is represented by the amino acid sequence as represented in FIG. 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or antigenic part thereof.

15. The composition of claim 1, wherein said composition further comprises Bacille Calmette Guerin [BCG].

16. The composition of claim 1, wherein said monoclonal antibody is an isotype selected from the group consisting of: IgA, IgM, IgD, IgE and IgG.

17. The composition according to claim 16, wherein said isotype is selected from the group consisting of: IgG1, IgG2, IgG3 and IgG4.

18. The composition according to claim 17, wherein said isotype is human IgG2 or IgG4.

19. The composition according to claim 16, wherein said antibody is a modified antibody wherein said modification reduces or abrogates the binding of said antibody to the B-cell receptor Fc gamma R Ilb.

20. The composition according to claim 19, wherein said modified antibody is an IgG antibody modified at C-g-2 domain of the heavy chain at asparagine 297 by deletion or substitution of said asparagine residue.

21. The composition according to claim 19, wherein said modified antibody is an IgG antibody modified at C-g-2 domain of the heavy chain at asparagine 265 by deletion or substitution of said asparagine residue.

22. The composition according to claim 19, wherein said modified antibody is an antibody modified at C-g-2 domain of the heavy chain at proline 331 by substitution with a serine residue.

23.-32. (canceled)

33. A method to treat a subject that is infected with or has a predisposition to a mycobacterial infection comprising administering to said subject an effective amount of a vaccine composition wherein said composition comprises a nucleic acid molecule or polypeptide selected from the group consisting of:
i) a nucleic acid molecule as represented by the nucleic acid sequence in FIG. 1a and/or FIG. 2a and/or FIG. 3a and/or FIG. 4a and/or FIG. 5a and/or FIG. 6a and/or FIG. 7a and/or FIG. 8a and/or FIG. 9a;
ii) a nucleic acid molecule that hybridizes to the nucleic acid molecule in (i) under stringent hybridization conditions wherein said nucleic acid encodes a polypeptide that has the activity associated with the mycobacterial proteins Ag85A, Ag85B, AgAg85C, GroEL, GroEL 2, GroES, PSTS3, TB10.4 and ESAT-6; and
iii) a polypeptide comprising an amino acid sequence as represented in FIGS. 1b and/or FIG. 2b and/or FIG. 3b and/or FIG. 4b and/or FIG. 5b and/or FIG. 6b and/or FIG. 7b and/or FIG. 8b and/or FIG. 9b or antigenic part thereof; and wherein said composition further comprises a nucleic acid molecule that encodes a CD40 ligand, or a polypeptide with CD40 ligand binding activity that binds and activates CD40 receptor expressed by antibody producing B-lymphocytes.

34.-51. (canceled)


SEQUENCE LISTING (59 sequences)

SEQ ID NO: 1 - DNA, 1017 bp, Mycobacterium tuberculosis
ctaggcgccctggggcgcgggcccggtgttgggcgtggcacccagtgcccgttgcaggtcgggcttcatagcgttgagctgcgcgccccagtactcccagctgtgcgtaccgctgtccgggaagtcgaacacgccgttgtggccgccaccggcgttgtaggcgtcttggaacttgatgttgctggtccgcacgaagccctcgaggaacttggccggcaggttgttgccacccagatccgacggcttgccgttgccgcagtacacccagacgcgggtgttgttggcgatcagcttcccgacgttcaacagcgggtcgttgcgctgccacgccgggtcctccttcgggccccacatgtcggaggccttgtagccgccagcgtcacccatcgccaggccgatcagggtgggacccatcgcctgggaggggtccaacaggcccgacatcgctcccgcgtagacgaactgctgggggtgatagatcgccagcgtcagcgccgaagaagcagccatcgaaagaccgacgacggcgcttccggtgggcttgacgtgcctgttggcctgcagccaccccggcagctcgctggtcaggaaggtctcccacttgtaagtctggcaaccggccttgccgcaggcgggctggtaccagtcggagtagaagcttgactggccacccaccggcatgaccaccgacaggcccgactggtcgtaccactcgaacgccggggtgttgatgtcccagccgctgaagtcgtcctgcgcgcgcaggccgtcgagcaggtacagggcgggcgagttggcaccaccactttggaattggaccttgatgtcacggcccatcgacggcgacggcacctgcaggtactccaccggcaagcccggccgggaaaatgcccccgcggtcgccgtgccaccgacggcgccgaccagacccgacactagggccgcgccgacggccccgaccacgagtcgacgcgacatacccgtgacggcgccacgaaccctgtcaacaagctgcat

SEQ ID NO: 2 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 3 - DNA, 978 bp, Mycobacterium tuberculosis
tcagccggcgcctaacgaactctgcaggtcacccttcatggcgttgagctgagcgccccagtactcccagctgtgcgtgccgttgggcgggaagttgaacacggcgttgtgcccgcccgcggcgttgtacgcatcctggaacttcaggttgctgctacgaacgaagttctccaagaactcggcgggtatgttggcaccgcccaactcgttcggggtgccgttcccgcaataaacccatagccgggtgttgtttgcgaccagcttggggatctgctgcgtagggtcgttgcgctcccatgccgggtcactcgagggaccccacatgtctgcggccttgtaaccgccggcgtcacccatcgcgaggccgatcaggctaggccccatcccctgagaggggtccagcagggccgacagcgagccggcgtagatgaactgctgggggtggtaggcggccaagatcattgccgacgagccggccatcgacaagccgattgcagcgctgccggtgggcttcacggccctgttggcggacaaccattgcggcagctcgctggtcaggaaggtttcccacttgtaagtctggcagccagccttaccgcaggccgggctgtaccagtcgctgtagaagctggactgcccgccgaccggcatgactatcgacagtcccgactggtagtaccactcgaacgccggggtgttgatatcccagccgttgtagtcgtcttgggcgcgcaggccgtcgagcagataaaccgcaggtgagttgttcccaccgctctggaactgaaccttgatgtcgcggcccatcgacggcgacggcacctgcaggtactcgaccggcagccccggccgggagaacgcgcccgcggttgccgctccgccggcaagccccaccaggcccggaaggactacagccgctgccgtgccgatcatcaatcggcgtccccaagctcgaatctttcggctcacgtctgtcat

SEQ ID NO: 4 - protein, 324 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSNLKFQDAYNAAGGHNAVFNFPPNGTHSWEYWGAQLNAMKGDLQSSLGAG

SEQ ID NO: 5 - DNA, 900 bp, Mycobacterium tuberculosis
ttagcggatcgcaccgacgatatcgcccgacatagcgcccagctggggcgcccacgagccccagccgttgtcaccgctggctgggaagtcgaagtgtccgttgtgcccgccgacgctgcgatactggttgtagaacatgcggctgttacccatcgcctcggcggcttggccgatcatggcggcgggatcgctggctcccgggttggtcgggctccacacccacacccgggtgttgttttgcgccagcaggctggcatgcacccacgggtcgtgccacttccaccgacccagctgtggtgctccccacattccgttggtgtccacaccgccgaattgctgcatgcccgccgcgatcgcaccgttggtggtggtgttcgacgggtacaaaaagcccgacatcgagccagcgaagccgaagcggtcggggtggaaggccgccagcgccatcgccccgtaaccgccctgagcggcgccaacggccgcatggccaccgggggccaagccccggttagcggccagccagtcgggcagctcagcggacaagaaggtgtcccactgcttgctgccatcctgctcccagttggtgtacatgctgtacgcaccaccggccggtgccaccaccgaaatccccttgcccgccaacgtgttcatcgcgttacccgcggtgacccagttactgacatccgggccggcgttgaaggcgtccagcagatacaccgcgtgcggcccaccggctaggaaggccaccgggatgtcccggcccatcgagggcgacggcaccatcaggttctcgtatggggcggccttggcggtgggttccgcggctaccgcgacaccgcccaacccgaatgacagtgcggcaatccagagcgcccgcagcagcgccgaccgacccttcat

SEQ ID NO: 6 - protein, 299 aa, Mycobacterium tuberculosis
MKGRSALLRALWIAALSFGLGGVAVAAEPTAKAAPYENLMVPSPSMGRDIPVAFLAGGPHAVYLLDAFNAGPDVSNWVTAGNAMNTLAGKGISVVAPAGGAYSMYTNWEQDGSKQWDTFLSAELPDWLAANRGLAPGGHAAVGAAQGGYGAMALAAFHPDRFGFAGSMSGFLYPSNTTTNGAIAAGMQQFGGVDTNGMWGAPQLGRWKWHDPWVHASLLAQNNTRVWVWSPTNPGASDPAAMIGQAAEAMGNSRMFYNQYRSVGGHNGHFDFPASGDNGWGSWAPQLGAMSGDIVGAIR

SEQ ID NO: 7 - DNA, 1620 bp, Mycobacterium tuberculosis
tcagtgcgcgtgcccgtggtgatggtcgtgatcttctgccttggccggcttgtcgaccacgaccgtctcggtggtgagtaccatccgggcaaccgatgacgcgttcaacaccgccgacctagtcaccttgaccgggtcgatgacgccgtcagcggccaagtcaccatagctcagggtgttcacgttcagcccatgcccggcgggtagctcgctgaccttgttgaccaccaccgagccgtccaagccagcgttggcggcgatccagaacaacggcgcggcaagggcttcggagaacacgtcgacaccgaggacctcgtcaccggtcagcgacgcacgcagttcggtcagcgccttgcgggcctggtggatgagcgaggctcccccaccagggacgatgccctcctcgaccgcggccttggcggccgcgaccgcatcctcgacgctttccttgcgctccttgagtgcggtctcggtggcggcacccaccttgatgacagcaaccccgccggccagtttggccagccgctcgccaagcttttcccgatcccaatccgaatcgctcttgtcgatctcggcacgcaagtgcttcgcccggttggccaccgcttctgcggtgccgccgccgtcgacaatgaccgtgtcgtccttgctgaccaccacgcgtcgggccgagcccagcacctccaagcccacctcgcgcagcaccatgccggcgtcggggttgaccacctggccacccgtcaccaccgccaggtcctcaaggaacgccttacggcggtcaccgaagtacggccccttgaccgcgaccgctttcaacgtcttgcgaatcgcgttgacgaccagcgtcgccaacgcttcgccctccacgtcttcagccacgatcagtagtggcttacccgttcctgcaaccttttccagcaatggcaacagatcgggaagcgagctgatcttgtcttggtgcagcaggatcaacgcgtcctcgagcaccgcctgctggttatcgaagtcggtaacgaagtatgccgacaagaagcccttgtcgaagccgataccctcggtgaactccaactcggtgcccagcgtcgaggattcttcgacgctgaccacgccgtcgtggccgaccttgctcatcgcttcgccaaccaggtcaccgatctgctcgtcgcgcgaggacaccgtcgccacctgcgcgatgccggtcttgccggacaccggcgtggccgatgccagcagcgcctcggataccgcgtcggcggccttgccgattcccacgccgagcgcgatcgggttgacgccggcggccactagcctcaggccgcccttgatcagtgcctgcgccaagatggttgcggtggtggtgccgtcaccggccacatcgttggtcttggtggccaccgacttcaccagctgggcgcccaagtcttcaaacggatcttccagctcgatctcacgtgccaccgtgacgccgtcgttggtaaccgtgggtccgccaaacgccttggccagcaccacatgccggccgcgcggccccagcgtcacccgcacggtgtcggccagcttgtccatgccgacctccatggcgcgacgcgcggtttcgtcgtattcgatcagcttgctcat

SEQ ID NO: 8 - protein, 539 aa, Mycobacterium tuberculosis
MSKLIEYDETARRAMEVGMDKLADTVRVTLGPRGRHVVLAKAFGGPTVTNDGVTVAREIELEDPFEDLGAQLVKSVATKTNDVAGDGTTTATILAQALIKGGLRLVAAGVNPIALGVGIGKAADAVSEALLASATPVSGKTGIAQVATVSSRDEQIGDLVGEAMSKVGHDGVVSVEESSTLGTELEFTEGIGFDKGFLSAYFVTDFDNQQAVLEDALILLHQDKISSLPDLLPLLEKVAGTGKPLLIVAEDVEGEALATLVVNAIRKTLKAVAVKGPYFGDRRKAFLEDLAVVTGGQVVNPDAGMVLREVGLEVLGSARRVVVSKDDTVIVDGGGTAEAVANRAKHLRAEIDKSDSDWDREKLGERLAKLAGGVAVIKVGAATETALKERKESVEDAVAAAKAAVEEGIVPGGGASLIHQARKALTELRASLTGDEVLGVDVFSEALAAPLFWIAANAGLDGSVVVNKVSELPAGHGLNVNTLSYGDLAADGVIDPVKVTRSAVLNASSVARMVLTTETVVVDKPAKAEDHDHHHGHAH

SEQ ID NO: 9 - DNA, 1623 bp, Mycobacterium tuberculosis
atggccaagacaattgcgtacgacgaagaggcccgtcgcggcctcgagcggggcttgaacgccctcgccgatgcggtaaaggtgacattgggccccaagggccgcaacgtcgtcctggaaaagaagtggggtgcccccacgatcaccaacgatggtgtgtccatcgccaaggagatcgagctggaggatccgtacgagaagatcggcgccgagctggtcaaagaggtagccaagaagaccgatgacgtcgccggtgacggcaccacgacggccaccgtgctggcccaggcgttggttcgcgagggcctgcgcaacgtcgcggccggcgccaacccgctcggtctcaaacgcggcatcgaaaaggccgtggagaaggtcaccgagaccctgctcaagggcgccaaggaggtcgagaccaaggagcagattgcggccaccgcagcgatttcggcgggtgaccagtccatcggtgacctgatcgccgaggcgatggacaaggtgggcaacgagggcgtcatcaccgtcgaggagtccaacacctttgggctgcagctcgagctcaccgagggtatgcggttcgacaagggctacatctcggggtacttcgtgaccgacccggagcgtcaggaggcggtcctggaggacccctacatcctgctggtcagctccaaggtgtccactgtcaaggatctgctgccgctgctcgagaaggtcatcggagccggtaagccgctgctgatcatcgccgaggacgtcgagggcgaggcgctgtccaccctggtcgtcaacaagatccgcggcaccttcaagtcggtggcggtcaaggctcccggcttcggcgaccgccgcaaggcgatgctgcaggatatggccattctcaccggtggtcaggtgatcagcgaagaggtcggcctgacgctggagaacgccgacctgtcgctgctaggcaaggcccgcaaggtcgtggtcaccaaggacgagaccaccatcgtcgagggcgccggtgacaccgacgccatcgccggacgagtggcccagatccgccaggagatcgagaacagcgactccgactacgaccgtgagaagctgcaggagcggctggccaagctggccggtggtgtcgcggtgatcaaggccggtgccgccaccgaggtcgaactcaaggagcgcaagcaccgcatcgaggatgcggttcgcaatgccaaggccgccgtcgaggagggcatcgtcgccggtgggggtgtgacgctgttgcaagcggccccgaccctggacgagctgaagctcgaaggcgacgaggcgaccggcgccaacatcgtgaaggtggcgctggaggccccgctgaagcagatcgccttcaactccgggctggagccgggcgtggtggccgagaaggtgcgcaacctgccggctggccacggactgaacgctcagaccggtgtctacgaggatctgctcgctgccggcgttgctgacccggtcaaggtgacccgttcggcgctgcagaatgcggcgtccatcgcggggctgttcctgaccaccgaggccgtcgttgccgacaagccggaaaaggagaaggcttccgttcccggtggcggcgacatgggtggcatggatttctga

SEQ ID NO: 10 - protein, 540 aa, Mycobacterium tuberculosis
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKGAKEVETKEQIAATAAISAGDQSIGDLIAEAMDKVGNEGVITVEESNTFGLQLELTEGMRFDKGYISGYFVTDPERQEAVLEDPYILLVSSKVSTVKDLLPLLEKVIGAGKPLLIIAEDVEGEALSTLVVNKIRGTFKSVAVKAPGFGDRRKAMLQDMAILTGGQVISEEVGLTLENADLSLLGKARKVVVTKDETTIVEGAGDTDAIAGRVAQIRQEIENSDSDYDREKLQERLAKLAGGVAVIKAGAATEVELKERKHRIEDAVRNAKAAVEEGIVAGGGVTLLQAAPTLDELKLEGDEATGANIVKVALEAPLKQIAFNSGLEPGVVAEKVRNLPAGHGLNAQTGVYEDLLAAGVADPVKVTRSALQNAASIAGLFLTTEAVVADKPEKEKASVPGGGDMGGMDF

SEQ ID NO: 11 - DNA, 303 bp, Mycobacterium tuberculosis
ctacttggaaacgacggccagcacgtcgcgtgccgacaggatcaggtattcctcgccgttgtacttgatctcggtgccgccgtacttgctgtagatgacggtgtcaccctccgcaacgtccagcgggatccgcttctcgccgtcctcgtcccaccggccagggccgacggcaacgacggtgccctcctgcggcttctccttggcggtgtcaggaatgaccagaccggacgcggtcgtggtctcggcctcgttggcctgcacgagaatcttgtcctcgagtggcttgatgttcaccttcgccac

SEQ ID NO: 12 - protein, 100 aa, Mycobacterium tuberculosis
MAKVNIKPLEDKILVQANEAETTTASGLVIPDTAKEKPQEGTVVAVGPGRWDEDGEKRIPLDVAEGDTVIYSKYGGTEIKYNGEEYLILSARDVLAVVSK

SEQ ID NO: 13 - DNA, 210 bp, Mycobacterium tuberculosis
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcag

SEQ ID NO: 14 - protein, 70 aa, Mycobacterium tuberculosis
MTEQQWNFAGIEAAASAIQGNVTSIHSLLDEGKQSLTKLAAAWGGSGSEAYQGVQQKWDATATELNNALQ

SEQ ID NO: 15 - DNA, 1113 bp, Mycobacterium tuberculosis
ttgaaactcaaccgatttggtgccgccgtaggtgtcctggctgcgggtgcgctggtgttgtccgcgtgtggtaacgacgacaatgtgaccgggggaggtgcaaccactggccaggcgtcggcgaaggtcgattgcggggggaagaagacactcaaagccagtgggtcgacggcgcaggccaacgcgatgacccgctttgtcaacgtgttcgagcaggcctgccccggccaaaccctgaactacacggccaatggttcgggcgctggaatcagcgaatttaatggcaaccaaaccgatttcggtggctcagatgtacccctgagcaaggacgaggccgcagcggcgcagcggcgttgcggctcgccggcgtggaatctgccggtggtgttcggcccgatcgcggttacctacaacctcaacagcgtttcctcgctaaatttggacggccccacgttggcgaagatcttcaacggctccattacgcagtggaacaatcccgcgatccaggcgctgaaccgcgacttcacgctgccaggtgagcggattcacgtggtgttccgcagcgatgagtcggggaccacggacaacttccagaggtacctgcaggccgcgtccaacggtgcgtggggtaagggcgctggaaagtcgttccaaggcggcgtcggtgagggcgcgcggggtaacgatggcacgtcagcggccgcgaagaacaccccggggtcgatcacctacaacgagtggtcgttcgcccaggcgcagcacctgaccatggccaacatcgtcacttcggctggtggggacccggtggcgattactatcgactcggtcggccagacgatcgccggggccaccatctccggggtgggcaacgacctggtgctcgacacggactcgttctaccggccgaagcgtcccggctcctatccgatcgtgttagcgacatacgaaatcgtttgctcgaagtatcccgactcgcaggttggcacggctgtgaaggcgttcctgcagagcactatcggcgccggtcaaagcggcctgggggacaacggatacatcccaattccggacgagttcaaatcgaggctgtcgactgcggtcaacgcgatcgcctga

SEQ ID NO: 16 - protein, 370 aa, Mycobacterium tuberculosis
MKLNRFGAAVGVLAAGALVLSACGNDDNVTGGGATTGQASAKVDCGGKKTLKASGSTAQANAMTRFVNVFEQACPGQTLNYTANGSGAGISEFNGNQTDFGGSDVPLSKDEAAAAQRRCGSPAWNLPVVFGPIAVTYNLNSVSSLNLDGPTLAKIFNGSITQWNNPAIQALNRDFTLPGERIHVVFRSDESGTTDNFQRYLQAASNGAWGKGAGKSFQGGVGEGARGNDGTSAAAKNTPGSITYNEWSFAQAQHLTMANIVTSAGGDPVAITIDSVGQTIAGATISGVGNDLVLDTDSFYRPKRPGSYPIVLATYEIVCSKYPDSQVGTAVKAFLQSTIGAGQSGLGDNGYIPIPDEFKSRLSTAVNAIA

SEQ ID NO: 17 - DNA, 291 bp, Mycobacterium tuberculosis
atgtcgcaaatcatgtacaactaccccgcgatgttgggtcacgccggggatatggccggatatgccggcacgctgcagagcttgggtgccgagatcgccgtggagcaggccgcgttgcagagtgcgtggcagggcgataccgggatcacgtatcaggcgtggcaggcacagtggaaccaggccatggaagatttggtgcgggcctatcatgcgatgtccagcacccatgaagccaacaccatggcgatgatggcccgcgacacggccgaagccgccaaatggggcggctag

SEQ ID NO: 18 - protein, 96 aa, Mycobacterium tuberculosis
MSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 19 - protein, 373 aa, Mycobacterium tuberculosis
RSERLRWLVAAEGPFASVYFDDSHDTLDAVERREATWRDVRKHLESRDAKQELIDSLEEAVRDSRPAVGQRGRALIATGEQVLVNEHLIGPPPATVIRLSDYPYVVPLIDLEMRRPTYVFAAVDHTGADVKLYQGATISSTKIDGVGYPVHKPVTAGWNGYGDFQHTTEEAIRMNCRAVADHLTRLVDAADPEVVFVSGEVRSRTDLLSTLPQRVAVRVSQLHAGPRKSALDEEEIWDLTSAEFTRRRYAEITNVAQQFEAEIGRGSGLAAQGLAEVCAALRDGDVDTLIVGELGEATVVTGKARTTVARDADMLSELGEPVDRVARADEALPFAAIAVGAALVRDDNRIAPLDGVGALLRYAATNRLGSHRS

SEQ ID NO: 20 - protein, 80 aa, Mycobacterium tuberculosis
MTNVGDQGVDAVFGVIYPPQVALVSFGKPAQRVCAVDGAIHVMTTVLATLPADHGCSDDHRGALFFLSINELTRCAAVTG

SEQ ID NO: 21 - protein, 165 aa, Mycobacterium tuberculosis
MGATAITVLAGAHIVEMADAPMAIVTSGLVAGASVVFWAFGPWLIPPLVAASIWKHVVHRVPLRYEATLWSVVFPLGMYGVGAYRLGLAAHLPIVESIGEFEGWVALAVWTITFVAMLHHLAATIGRSGRSSHAIGAADDTHAIICRPPRSFDHQVRAFRRNQPM

SEQ ID NO: 22 - protein, 652 aa, Mycobacterium tuberculosis
MTVTPRTGSRIEELLARSGRFFIPGEISADLRTVTRRGGRDGDVFYRDRWSHDKVVRSTHGVNCTGSCSWKIYVKDDIITWETQETDYPSVGPDRPEYEPRGCPRGAAFSWYTYSPTRVRHPYARGVLVEMYREAKARLGDPVAAWADIQADPRRRRRYQRARGKGGLVRVSWAEATEMIAAAHVHTISTYGPDRVAGFSPIPAMSMVSHAAGSRFVELIGGVMTSFYDWYADLPVASPQVFGDQTDVPESGDWWDVVWQCASVLLTYPNSRQLGTAEELLAHIDGPAADLLGRTVSELRRADPLTAATRYVDTFDLRGRATLYLTYWTAGDTRNRGREMLAFAQTYRSTDVAPPRGETPDFLPVVLEFAATVDPEAGRRLLSGYRVPIAALCNALTEAALPYAHTVAAVCRTGDMMGELFWTVVPYVTMTIVAVGSWWRYRYDKFGWTTRSSQLYESRLLRIASPMFHFGILVVIVGHGIGLVIPQSWTQAAGLSEGAYHVQAVVLGSIAGITTLAGVTLLIYRRRTRGPVFMATTVNDKVMYLVLVAAIVAGLGATALGSGVVGEAYNYRETVSVWFRSVWVLQPRGDLMAEAPLYYQIHVLIGLALFALWPFTRLVHAFSAPIGYLFRPYIIYRSREELVLTRPRRRGW

SEQ ID NO: 23 - protein, 395 aa, Mycobacterium tuberculosis
MRGQAANLVLATWISVVNFWAWNLIGPLSTSYARDMSLSSAEASLLVATPILVGALGRIVTGPLTDRFGGRAMLIAVTLASILPVLAVGVAATMGSYALLVFFGLFLGVAGTIFAVGIPFANNWYQPARRGFSTGVFGMGMVGTALSAFFTPRFVRWFGLFTTHAIVAAALASTAVVAMVVLRDAPYFRPNADPVLPRLKAAARLPVTWEMSFLYAIVFGGFVAFSNYLPTYITTIYGFSTVDAGARTAGFALAAVLARPVGGWLSDRIAPRHVVLASLAGTALLAFAAALQPPPEVWSAATFITLAVCLGVGTGGVFAWVARRAPAASVGSVTGIVAAAGGLGGYFPPLVMGATYDPVDNDYTVGLLLLVATALVACTYTALHAREPVSEEASR

SEQ ID NO: 24 - protein, 400 aa, Mycobacterium tuberculosis
MTRVVVIGSGFAGLWAALGAARRLDELAVLAGTVDVMVVSNKPFHDIRVRNYEADLSACRIPLGDVLGPAGVAHVTAEVTAIDADGRRVTTSTGASYSYDRLVLASGSHVVKPALPGLAEFGFDVDTYDGAVRLQQHLQGLAGGPLTSAAATVVVVGAGLTGIETACELPGRLHALFARGDGVTPRVVLIDHNPFVGSDMGLSARPVIEQALLDNGVETRTGVSVAAVSPGGVTLSSGERLAAATVVWCAGMRASRLTEQLPVARDRLGRLQVDDYLRVIGVPAMFAAGDVAAARMDDEHLSVMSCQHGRPMGRYAGCNVINDLFDQPLLALRIPWYVTVLDLGSAGAVYTEGWERKVVSQGAPAKTTKQSINTRRIYPPLNGSRADLLAAAAPRVQPRP

SEQ ID NO: 25 - protein, 905 aa, Mycobacterium tuberculosis
MSASVSATTAHHGLPAHEVVLLLESDPYHGLSDGEAAQRLERFGPNTLAVVTRASLLARILRQFHHPLIYVLLVAGTITAGLKEFVDAAVIFGVVVINAIVGFIQESKAEAALQGLRSMVHTHAKVVREGHEHTMPSEELVPGDLVLLAAGDKVPADLRLVRQTGLSVNESALTGESTPVHKDEVALPEGTPVADRRNIAYSGTLVTAGHGAGIVVATGAETELGEIHRLVGAAEVVATPLTAKLAWFSKFLTIAILGLAALTFGVGLLRRQDAVETFTAAIALAVGAIPEGLPTAVTITLAIGMARMAKRRAVIRRLPAVETLGSTTVICADKTGTLTENQMTVQSIWTPHGEIRATGTGYAPDVLLCDTDDAPVPVNANAALRWSLLAGACSNDAALVRDGTRWQIVGDPTEGAMLVVAAKAGFNPERLATTLPQVAAIPFSSERQYMATLHRDGTDHVVLAKGAVERMLDLCGTEMGADGALRPLDRATVLRATEMLTSRGLRVLATGMGAGAGTPDDFDENVIPGSLALTGLQAMSDPPRAAAASAVAACHSAGIAVKMITGDHAGTATAIATEVGLLDNTEPAAGSVLTGAELAALSADQYPEAVDTASVFARVSPEQKLRLVQALQARGHVVAMTGDGVNDAPALRQANIGVAMGRGGTEVAKDAADMVLTDDDFATIEAAVEEGRGVFDNLTKFITWTLPTNLGEGLVILAAIAVGVALPILPTQILWINMTTAIALGLMLAFEPKEAGIMTRPPRDPDQPLLTGWLVRRTLLVSTLLVASAWWLFAWELDNGAGLHEARTAALNLFVVVEAFYLFSCRSLTRSAWRLGMFANRWIILGVSAQAIAQFAITYLPAMNMVFDTAPIDIGVWVRIFAVATAITIVVATDTLLPRIRAQPP

SEQ ID NO: 26 - protein, 257 aa, Mycobacterium tuberculosis
SFHDLHHQGVPFVLPNAWDVPSALAYLAEGFTAIGTTSFGVSSSGGHPDGHRATRGANIALAAALAPLQCYVSVDIEDGYSDEPDAIADYVAQLSTAGINIEDSSAEKLIDPALAAAKIVAIKQRNPEVFVNARVDTYWLRQHADTTSTIQRALRYVDAGADGVFVPLANDPDELAELTRNIPCPVNTLPVPGLTIADLGELGVARVSTGSVPYSAGLYAAAHAARAVSDGEQLPRSVPYAELQARLVDYENRTSTT

SEQ ID NO: 27 - protein, 285 aa, Mycobacterium tuberculosis
MVKRSRATRLSPSIWSGWESPQCRSIRARLLLPRGRSRPPNADCCWNQLAVTPDTRMPASSAAGRDAAAYDAWYDSPTGRPILATEVAALRPLIEVFAQPRLEIGVGTGRFADLLGVRFGLDPSRDALMFARRRGVLVANAVGEAVPFVSRHFGAVLMAFTLCFVTDPAAIFRETRRLLADGGGLVIGFLPRGTPWADLYALRAARGQPGYRDARFYTAAELEQLLADSGFRVIARRCTLHQPPGLARYDIEAAHDGIQAGAGFVAISAVDQAHEPKDDHPLESE

SEQ ID NO: 28 - protein, 498 aa, Mycobacterium tuberculosis
MDSPTNDGTCDAHPVTDEPFIDVRETHTAVVVLAGDRAFKAKKPVVTDFCDFRTAEQRERACIREFELNSRLAAQSYLGIAHLSDPSGGHAEPVVVMRRYRDKQRLASMVTAGLPVEGALDAIAEVLARFHQRAQRNRCIDTQGEVGAVARRWHENLAELRHHADKVVSGDVIRRIEHMVDEFVSGREVLFAGRIKEGCIVDGHADLLADDIFLVDGEPALLDCLEFEDELRYLDRIDDAAFLAMDLEFLGRKDLGDYFLAGYAVRSGDTAPASLRDFYIAYRAVVRAKVECVRFSQGKPEAAADAVRHLIIATQHLQHATVRLALVGGNPGTGKSTLARGVAELVGAQVISTDDVRRRLRDCGVITGEPGVLDSGLYSRANVVAVYQEALRKARLLLGSGHSVILDGTWGDPQMRACARRLAADTHSAIVEFRCSATVDVMADRIVARAGGNSDATAEIAAALAARQADWDTGHRIDTAGPRERSVGQAYHIWRSAI

SEQ ID NO: 29 - protein, 295 aa, Mycobacterium tuberculosis
MSKPRKQHGVVVGVDGSLESDAAACWGATDAAMRNIPLTVVHVVNADVATWPPMPYPETWGVWQEDEGRQIVANAVKLAKEAVGADRKLSVKSELVFSTPVPTMVEISNEAEMVVLGSSGRGALARGLLGSVSSSLVRRAGCPVAVIHSDDAVIPDPQHAPVLVGIDGSPVSELATAVAFDEASRRGVELIAVHAWSDVEVVELPGLDFSAVQQEAELSLAERLAGWQERYPDVPVSRVVVCDRPARKLVQKSASAQLVVVGSHGRGGLTGMLLGSVSNAVLHAARVPVIVARQS

SEQ ID NO: 30 - protein, 279 aa, Mycobacterium tuberculosis
MNQSHKPPSIVVGIDGSKPAVQAALWAVDEAASRDIPLRLLYAIEPDDPGYAAHGAAARKLAAAENAVRYAFTAVEAADRPVKVEVEITQERPVTSLIRASAAAALVCVGAIGVHHFRPERVGSTAAALALSAQCPVAIVRPHRVPIGRDAAWIVVEADGSSDIGVLLGAVMAEARLRDSPVRVVTCRQSGVGDTGDDVRASLDRWLARWQPRYPDVRVQSAAVHGELLDYLAGLGRSVHMVVLSASDQEHVEQLVGAPGNAVLQEAGCTLLVVGQQYL

SEQ ID NO: 31 - protein, 392 aa, Mycobacterium tuberculosis
MRDAIPLGRIAGFVVNVHWSVLVILWLFTWSLATMLPGTVGGYPAVVYWLLGAGGAVMLLASLLAHELAHAVVARRAGVSVESVTLWLFGGVTALGGEAKTPKAAFRIAFAGPATSLALSATFGALAITLAGVRTPAIVISVAWWLATVNLLLGLFNLLPGAPLDGGRLVRAYLWRRHGDSVRAGIGAARAGRVVALVLIALGLAEFVAGGLVGGVWLAFIGWFIFAAAREEETRISTQQLFAGVRVADAMTAQPHTAPGWINVEDFIQRYVLGERHSAYPVADRDGSITGLVALRQLRDVAPSRRSTTSVGDIALPLHSVPTARPQEPLTALLERMAPLGPRSRALVTEGSAVVGIVTPSDVARLIDVYRLAQPEPTFTTSPQDADRFSDA

SEQ ID NO: 32 - protein, 179 aa, Mycobacterium tuberculosis
MLHRDDHINPPRPRGLDVPCARLRATNPLRALARCVQAGKPGTSSGHRSVPHTADLRIEAWAPTRDGCIRQAVLGTVESFLDLESAHAVHTRLRRLTADRDDDLLVAVLEEVIYLLDTVGETPVDLRLRDVDGGVDVTFATTDASTLVQVGAVPKAVSLNELRFSQGRHGWRCAVTLDV

SEQ ID NO: 33 - protein, 432 aa, Mycobacterium tuberculosis
MQVVNVATLPGIVRASYAMPDVHWGYGFPIGGVAATDVDNDGVVSPGGVGFDISCGVRLLVGEGLDREELQPRLPAVMDRLDRAIPRGVGTAGVWRLPDRNTLQEVLTGGARFAVEQGHGVALDLERCEDGGVMTGADAAKISDRALQRGLGQIGSLGSGNHFLEVQAVDRVYDPVAAAPMGLAEGTVCVMIHTGSRGLGHQICTDHVRQMEQAMGRYGIAVPDRQLACVPVHSPDGQAYLAAMAAAANYGRANRQLLTEATRRVFADATGTPLDLLYDVSHNLAKIETHPIDGQLRSVCVHRKGATRSLPPHHHELPAELAAVGQPVLIPGTMGTASYVLAGVTGNPAFFSTAHGAGRVLSRHQAARHTSGEAIRASLAKRGIIVRGTSRRGIAEEKPEAYKDVDEVIEASHQSGLARKVARLVPLGCVKG

SEQ ID NO: 34 - protein, 336 aa, Mycobacterium tuberculosis
MWSASGGQCGKYLAASMVLQLDGLERHGVLEFGRDRYGPEVREELLAMSAASIDRYLKTAKAKDQISGVSTTKPSPLLRNSIKVRRAGDEVEAEPGFFEGDTVAHCGPTLKGEFAHTLNLTDVHIGWVFTRTVRNNARTHILAGLKASVTEIPHGITGLDFDNGTVFLNKPVISWAGDNGIYFTRFRPYKKNHATIESKNNHLVRKYAFYYRYDTAEERAVLNRMWKLVNDRLNYLTPTIKPIGYASSADGRRRRLYDAPQTPLDRPLAARVLSAAQQADLITYRDSLNPAQIGRKIADLQNRLLILAKEKTEQLYLANIPTALPDIHKGILIKAG

SEQ ID NO: 35 - protein, 273 aa, Mycobacterium tuberculosis
VEPKRSRLVVCAPEPSHAREFPDVAVFSGGRANASQAERLARAVGRVLADRGVTGGARVRLTMANCADGPTLVQINLQVGDTPLRAQAATAGIDDLRPALIRLDRQIVRASAQWCPRPWPDRPRRRLTTPAEALVTRRKPVVLRRATPLQAIAAMDAMDYDVHLFTDAETGEDAVVYRAGPSGLRLARQHHVFPPGWSRCRAPAGPPVPLIVNSRPTPVLTEAAAVDRAREHGLPFLFFTDQATGRGQLLYSRYDGNLGLITPTGDGVADGLA

SEQ ID NO: 36 - protein, 88 aa, Mycobacterium tuberculosis
MKAKVGDWLVIKGATIDQPDHRGLIIEVRSSDGSPPYVVRWLETDHVATVIPGPDAVVVTAEEQNAADERAQHRFGAVQSAILHARGT

SEQ ID NO: 37 - protein, 113 aa, Mycobacterium tuberculosis
MGEHAIKRHMRQRKPTKHPLAQKRGARILVFTDDPRRSVLIVPGCHLDSMRREKNAYYFQDGNALVGMVVSGGTVEYDADDRTYVVQLTDGRHTTESSFEHSSPSRSPQSDDL

SEQ ID NO: 38 - protein, 210 aa, Mycobacterium tuberculosis
MIATTRDREGATMITFRLRLPCRTILRVFSRNPLVRGTDRLEAVVMLLAVTVSLLTIPFAAAAGTAVQDSRSHVYAHQAQTRHPATATVIDHEGVIDSNTTATSAPPRTKITVPARWVVNGIERSGEVNAKPGTKSGDRVGIWVDSAGQLVDEPAPPARAIADAALAALGLWLSVAAVAGALLALTRAILIRVRNASWQHDIDSLFCTQR

SEQ ID NO: 39 - protein, 94 aa, Mycobacterium tuberculosis
MCGDQSDHVLQHWTVDISIDEHEGLTRAKARLRWREKELVGVGLARLNPADRNVPEIGDELSVARALSDLGKRMLKVSTHDIEAVTHQPARLLY

SEQ ID NO: 40 - protein, 143 aa, Mycobacterium tuberculosis
MITNLRRRTAMAAAGLGAALGLGILLVPTVDAHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACN

SEQ ID NO: 41 - protein, 317 aa, Mycobacterium tuberculosis
MSAQQTNLGIVVGVDGSPCSHTAVEWAARDAQMRNVALRVVQVVPPVITAPEGWAFEYSRFQEAQKREIVEHSYLVAQAHQIVEQAHKVALEASSSGRAAQITGEVLHGQIVPTLANISRQVAMVVLGYRGQGAVAGALLGSVSSSLVRHAHGPVAVIPEEPRPARPPHAPVVVGIDGSPTSGLAAEIAFDEASRRGVDLVALHAWSDMGPLDFPRLNWAPIEWRNLEDEQEKMLARRLSGWQDRYPDVVVHKVVVCDRPAPRLLELAQTAQLVVVGSHGRGGFPGMHLGSVSRAVVNSGQAPVIVARIPQDPAVPA

SEQ ID NO: 42 - protein, 114 aa, Mycobacterium tuberculosis
VTYVIGSECVDVMDKSCVQECPVDCIYEGARMLYINPDECVDCGACKPACRVEAIYWEGDLPDDQHQHLGDNAAFFHQVLPGRVAPLGSPGGAAAVGPIGVDTPLVAAIPVECP

SEQ ID NO: 43 - protein, 339 aa, Mycobacterium tuberculosis
MTEPAAWDEGKPRIITLTMNPALDITTSVDVVRPTEKMRCGAPRYDPGGGGINVARIVHVLGGCSTALFPAGGSTGSLLMALLGDAGVPFRVIPIAASTRESFTVNESRTAKQYRFVLPGPSLTVAEQEQCLDELRGAAASAAFVVASGSLPPGVAADYYQRVADICRRSSTPLILDTSGGGLQHISSGVFLLKASVRELRECVGSELLTEPEQLAAAHELIDRGRAEVVVVSLGSQGALLATRHASHRFSSIPMTAVSGVGAGDAMVAAITVGLSRGWSLIKSVRLGNAAGAAMLLTPGTAACNRDDVERFFELAAEPTEVGQDQYVWHPIVNPEASP

SEQ ID NO: 44 - protein, 681 aa, Mycobacterium tuberculosis
VLMTAAADVTRRSPRRVFRDRREAGRVLAELLAAYRDQPDVIVLGLARGGLPVAWEVAAALHAPLDAFVVRKLGAPGHDEFAVGALASGGRVVVNDDVVRGLRITPQQLRDIAEREGRELLRRESAYRGERPPTDITGKTVIVVDDGLATGASMFAAVQALRDAQPAQIVIAVPAAPESTCREFAGLVDDVVCATMPTPFLAVGESFWDFRQVTDEEVRRLLATPTAGPSLRRPAASTAADVLRRVAIDAPGGVPTHEVLAELVGDARIVLIGESSHGTHEFYQARAAMTQWLIEEKGFGAVAAEADWPDAYRVNRYVRGLGEDTNADEALSGFERFPAWMWRNTVVRDFVEWLRTRNQRYESGALRQAGFYGLDLYSLHRSIQEVISYLDKVDPRAAARARARYACFDHACADDGQAYGFAAAFGAGPSCEREAVEQLVDVQRNALAYARQDGLLAEDELFYAQQNAQTVRDAEVYYRAMFSGRVTSWNLRDQHMAQTLGSLLTHLDRHLDAPPARIVVWAHNSHVGDARATEVWADGQLTLGQIVRERYGDESRSIGFSTYTGTVTAASEWGGIAQRKAVRPALHGSVEELFHQTADSFLVSARLSRDAEAPLDVVRLGRAIGVVYLPATERQSHYLHVRPADQFDAMIHIDQTRALEPLEVTSRWIAGENPETYPTGL

SEQ ID NO: 45 - protein, 144 aa, Mycobacterium tuberculosis
MATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTN

SEQ ID NO: 46 - protein, 331 aa, Mycobacterium tuberculosis
MPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHR

SEQ ID NO: 47 - protein, 297 aa, Mycobacterium tuberculosis
MSSGNSSLGIIVGIDDSPAAQVAVRWAARDAELRKIPLTLVHAVSPEVATWLEVPLPPGVLRWQQDHGRHLIDDALKVVEQASLRAGPPTVHSEIVPAAAVPTLVDMSKDAVLMVVGCLGSGRWPGRLLGSVSSGLLRHAHCPVVIIHDEDSVMPHPQQAPVLVGVDGSSASELATAIAFDEASRRNVDLVALHAWSDVDVSEWPGIDWPATQSMAEQVLAERLAGWQERYPNVAITRVVVRDQPARQLVQRSEEAQLVVVGSRGRGGYAGMLVGSVGETVAQLARTPVIVARESLT

SEQ ID NO: 48 - protein, 272 aa, Mycobacterium tuberculosis
MSGRGEPTMKTIIVGIDGSHAAITAALWGVDEAISRAVPLRLVSVIKPTHPSPDDYDRDLAHAERSLREAQSAVEAAGKLVKIETDIPRGPAGPVLVEASRDAEMICVGSVGIGRYASSILGSTATELAEKAHCPVAVMRSKVDQPASDINWIVVRMTDAPDNEAVLEYAAREAKLRQAPILALGGRPEELREIPDGEFERRVQDWHHRHPDVRVYPITTHTGIARFLADHDERVQLAVIGGGEAGQLARLVGPSGHPVFRHAECSVLVVRR

SEQ ID NO: 49 - protein, 143 aa, Mycobacterium tuberculosis
MTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALAS

SEQ ID NO: 50 - protein, 413 aa, Mycobacterium tuberculosis
MASSASDGTHERSAFRLSPPVLSGAMGPFMHTGLYVAQSWRDYLGQQPDKLPIARPTIALAAQAFRDEIVLLGLKARRPVSNHRVFERISQEVAAGLEFYGNRRWLEKPSGFFAQPPPLTEVAVRKVKDRRRSFYRIFFDSGFTPHPGEPGSQRWLSYTANNREYALLLRHPEPRPWLVCVHGTEMGRAPLDLAVFRAWKLHDELGLNIVMPVLPMHGPRGQGLPKGAVFPGEDVLDDVHGTAQAVWDIRRLLSWIRSQEEESLIGLNGLSLGGYIASLVASLEEGLACAILGVPVADLIELLGRHCGLRHKDPRRHTVKMAEPIGRMISPLSLTPLVPMPGRFIYAGIADRLVHPREQVTRLWEHWGKPEIVWYPGGHTGFFQSRPVRRFVQAALEQSGLLDAPRTQRDRSA

SEQ ID NO: 51 - protein, 120 aa, Mycobacterium tuberculosis
MSTQRPRHSGIRAVGPYAWAGRCGRIGRWGVHQEAMMNLAIWHPRKVQSATIYQVTDRSHDGRTARVPGDEITSTVSGWLSELGTQSPLADELARAVRIGDWPAAYAIGEHLSVEIAVAV

SEQ ID NO: 52 - protein, 104 aa, Mycobacterium tuberculosis
MVIRFDQIGSLVLSMKSLASLSFQRCLRENSSLVAALDRLDAAVDELSALSFDALTTPERDRARRDRDHHPWSRSRSQLSPRMAHGAVHQCQWPKAVWAVIDNP

SEQ ID NO: 53 - protein, 344 aa, Mycobacterium tuberculosis
VLKNAVLLACRAPSVHNSQPWRWVAESGSEHTTVHLFVNRHRTVPATDHSGRQAIISCGAVLDHLRIAMTAAHWQANITRFPQPNQPDQLATVEFSPIDHVTAGQRNRAQAILQRRTDRLPFDSPMYWHLFEPALRDAVDKDVAMLDVVSDDQRTRLVVASQLSEVLRRDDPYYHAELEWWTSPFVLAHGVPPDTLASDAERLRVDLGRDFPVRSYQNRRAELADDRSKVLVLSTPSDTRADALRCGEVLSTILLECTMAGMATCTLTHLIESSDSRDIVRGLTRQRGEPQALIRVGIAPPLAAVPAPTPRRPLDSVLQIRQTPEKGRNASDRNARETGWFSPP

SEQ ID NO: 54 - protein, 110 aa, Mycobacterium tuberculosis
VVQGRTVLFRTAEGAKLFSAVAKCAVAFEADDHNVAEGWSVIVKVRAQVLTTDAGVREAERAQLLPWTATLKRHCVRVIPWEITGRHFRFGPEPDRSQTFACEASSHNQR

SEQ ID NO: 55 - protein, 463 aa, Mycobacterium tuberculosis
MNHLTTLDAGFLKAEDVDRHVSLAIGALAVIEGPAPDQEAFLSSLAQRLRPCTRFGQRLRLRPFDLGAPKWVDDPDFDLGRHVWRIALPRPGNEDQLFELIADLMARRLDRGRPLWEVWVIEGLADSKWAILTKLHHCMADGIAATHLLAGLSDESMSDSFASNIHTTMQSQSASVRRGGFRVNPSEALTASTAVMAGIVRAAKGASEIAAGVLSPAASSLNGPISDLRRYSAAKVPLADVEQVCRKFDVTINDVALAAITESYRNVLIQRGERPRFDSLRTLVPVSTRSNSALSKTDNRVSLMLPNLPVDQENPLQRLRIVHSRLTRAKAGGQRQFGNTLMAIANRLPFPMTAWAVGLLMRLPQRGVVTVATNVPGPRRPLQIMGRRVLDLYPVSPIAMQLRTSVAMLSYADDLYFGILADYDVVADAGQLARGIEDAVARLVAISKRRKVTRRRGALSLVV

SEQ ID NO: 56 - protein, 332 aa, Mycobacterium tuberculosis
MNTHFPDAETVRTVLTLAVRAPSIHNTQPWRWRVCPTSLELFSRPDMQLRSTDPDGRELILSCGVALHHCVVALASLGWQAKVNRFPDPKDRCHLATIGVQPLVPDQADVALAAAIPRRRTDRRAYSCWPVPGGDIALMAARAARGGVMLRQVSALDRMKAIVAQAVLDHVTDEEYLRELTIWSGRYGSVAGVPARNEPPSDPSAPIPGRLFAGPGLSQPSDVLPADDGAAILALGTETDDRLARLRAGEAASIVLLTATAMGLACCPITEPLEIAKTRDAVRAEVFGAGGYPQMLLRVGWAPINADPLPPTPRRELSQVVEWPEELLRQRC

SEQ ID NO: 57 - protein, 578 aa, Mycobacterium tuberculosis
MTTGGLVDENDGAAMRPLRHTLSQLRLHELLVEVQDRVEQIVEGRDRLDGLVEAMLVVTAGLDLEATLRAIVHSATSLVDARYGAMEVHDRQHRVLHFVYEGIDEETVRRIGHLPKGLGVIGLLIEDPKPLRLDDVSAHPASIGFPPYHPPMRTFLGVPVRVRDESFGTLYLTDKTNGQPFSDDDEVLVQALAAAAGIAVANARLYQQAKARQSWIEATRDIATELLSGTEPATVFRLVAAEALKLTAADAALVAVPVDEDMPAADVGELLVIETVGSAVASIVGRTIPVAGAVLREVFVNGIPRRVDRVDLEGLDELADAGPALLLPLRARGTVAGVVVVLSQGGPGAFTDEQLEMMAAFADQAALAWQLATSQRRMRELDVLTDRDRIARDLHDHVIQRLFAIGLALQGAVPHERNPEVQQRLSDVVDDLQDVIQEIRTTIYDLHGASQGITRLRQRIDAAVAQFADSGLRTSVQFVGPLSVVDSALADQAEAVVREAVSNAVRHAKASTLTVRVKVDDDLCIEVTDNGRGLPDEFTGSGLTNLRQRAEQAGGEFTLASVPGASGTVLRWSAPLSQ

SEQ ID NO: 58 - protein, 217 aa, Mycobacterium tuberculosis
VVKVFLVDDHEVVRRGLVDLLGADPELDVVGEAGSVAEAMARVPAARPDVAVLDVRLPDGNGIELCRDLLSRMPDLRCLILTSYTSDEAMLDAILAGASGYVVKDIKGMELARAVKDVGAGRSLLDNRAAAALMAKLRGAAEKQDPLSGLTDQERTLLGLLSEGLTNKQIADRMFLAEKTVKNYVSRLLAKLGMERRTQAAVFATELKRSRPPGDGP

SEQ ID NO: 59 - protein, 268 aa, Mycobacterium tuberculosis
MSDPRPARAVVVGIDGSRAATHAALWAVDEAVNRDIPLRLVYVIDPSQLSAAGEGGGQSAARAALHDASRKVEATGQPVKIETEVLCGRPLTKLMQESRSAAMLCVGSVGLDHVRGRRGSVAATLAGSALCPVAVIHPSPAEPATTSQVSAVVAEVDNGVVLRHAFEEARLRGVPLRAVAVHAAETPDDVEQGSRLAHVHLSRRLAHWTRLYPEVRVDRAIAGGSACRHLAANAKPGQLFVADSHSAHELCGAYQPGCAVLTVRSANL